(19) United States
(12) Patent Application Publication
(10) Pub. No.: US 2019/0070216 A1
(43) Pub. Date: Mar. 7, 2019

(54) Methods of Treating Osteoarthritis

(71) Applicant: DREXEL UNIVERSITY, Philadelphia, PA (US)

(72) Inventors: Michele Marcolongo, Aston, PA (US); Katsiaryna Prudnikova, Huntingdon Valley, PA (US); Mary Mulcahey, Villanova, PA (US); Evan Phillips, Mohnton, PA (US)

(21) Appl. No.: 16/178,198

(22) Filed: Nov. 1, 2018

Related U.S. Application Data
(63) Continuation of application No. 15/694,434, filed on Sep. 1, 2017, now abandoned.
(60) Provisional application No. 62/382,892, filed on Sep. 2, 2016.

(51) Int. Cl. A61K 31/78; A61K 45/06

(52) U.S. Cl. CPC A61K 31/78; A61K 45/06

(57) ABSTRACT

In one aspect, the present invention relates to a new method of treating or osteoarthritis in a mammal in need thereof. In certain embodiments, the method comprises contacting at least one biomimetic proteoglycan with the affected joint of the mammal. The biomimetic proteoglycan comprises a core structure and at least one bristle. The biomimetic proteoglycan resists endogenous enzymatic degradation and integrates with the existing tissue matrix.

### CROSS-REFERENCE TO RELATED APPLICATIONS

This application is a continuation application of, and claims priority to, U.S. patent application Ser. No. 15/694,434, filed Sep. 1, 2017, which claims priority to U.S. Provisional Patent Application No. 62/382,892, filed Sep. 2, 2016, the entire disclosure of which is incorporated herein by reference.

### BACKGROUND OF THE INVENTION

Osteoarthritis (OA) is the most common form of arthritis, affecting millions of people worldwide. Although OA can damage any joint in the body, the disease most commonly affects hand joints, knees, hips, and spine. Patients with OA suffer from decreased lubrication and load distribution in their joints due in part to a reduction in molecular weight and concentration of hyaluronic acid (HA) and lubricin in the synovial fluid or joint space. In addition, cartilaginous extracellular matrix (ECM) components aggrecan and collagen II experience homeostatic imbalance as degradation outpaces their synthesis. This breakdown of aggrecan and its reduced concentration in the tissue leads to increased permeability of the cartilage extracellular matrix and reduced hydrostatic pressure in degenerated cartilage, with a consequent loss of its ability to sustain and redistribute mechanical loads, which in turn produces symptoms as pain, joint stiffness and loss of mobility.

Natural aggrecan is physically responsible in part for cartilage hydration and osmotic pressure, which leads to cartilage mechanical stability. The hydration is controlled by the negative, fixed charge density of the CS bristles of the aggrecan molecule, while the cartilage pressure is controlled by the hydrostatic pressure in combination with electrostatic interactions between the aggrecan molecules due to the 3D molecular configuration. Therefore, both the high fixed charge density and the 3D molecular bottle brush configuration are essential to the mechanical function of the natural aggrecan. Biologically, aggrecan sequesters growth factors and other biomolecules and may upregulate cell synthetic activity. The importance of aggrecan is well recognized in the field, with several strategies under investigation to prevent the enzymatic degradation of aggrecan by aggrecanase (which attacks the protein core of natural aggrecan) with enzyme-specific inhibitors, as well as inhibitors to the inflammatory pathway that leads to the upregulation of aggrecanases and matrix metalloproteinases (MMPs). These strategies may serve to stop degeneration, but are not able to replenish the lost aggrecan molecules, since the biosynthesis of macromolecules by chondrocytes present in adult human tissue is minimal. Alternate strategies have been investigated to upregulate aggrecan cellular synthesis to potentially restore the matrix and overall mechanical stability by the introduction of growth factors, genetic engineering, and tissue engineering.

Although the lubrication mechanisms in articular cartilage have been studied for decades, and great advances in understanding of this complex behavior have been made, there are still uncertainties about the guiding principles of lubrication. There are different components contributing to joint lubrication thereby enabling joints articulate pain-free. Mechanically, cartilage needs to exhibit sufficient osmotic pressure, which is governed by proteoglycans, especially aggrecan and glycosaminoglycans. This allows the cartilage to draw water from surrounding tissue, thereby maintaining an important level of hydration necessary for the normal tribological behavior of the joint. Synovial fluid is complex, being composed of synovial cells, HA, lubricin and other key components. HA is responsible, in part, for hydration as well as viscosity of the synovial fluid. Viscosity is important for damping the impact loads experienced by the joint, so that cartilage is not compromised. Lubricin is a glycoprotein that is bound to the cartilage surface as a bottle-brush macromolecule. Lubricin provides hydration to the surface through negatively charged oligosaccharide bristles and helps maintain a boundary layer that aids in reducing joint friction. Additionally, there is an interaction between the cartilage and synovial fluid for joints in motion. Contact between the joint surfaces enables pressurization that permits boundary layer fluid motion, while unloaded regions of the joint are then able to resorb more moisture, due to the osmotic pressure in the cartilage from the negatively charged molecules: aggrecan and HA.

With osteoarthritis, there are molecular changes to the joint that have mechanical and tribological (friction and wear) consequences. In cartilage, aggrecan concentrations are reduced, enzymatically cleaved by MMPs and A Disintegrin And Metalloproteinase with Thrombospondin Motifs (ADAMTs) that attack the protein core of the molecule and result in reduced osmotic pressure of the cartilage. In synovial fluid, hyaluronidase is upregulated and cleaves the long HA molecules into shorter segments. Overall, the amount of HA in the synovial fluid is reduced. Likewise, lubricin is also compromised. Aggrecan serves an important role in the joint by allowing fluid to be pumped between cartilage and synovial fluid and to maintain contact pressure. The lubricating quality of synovial fluid is unchanged after digesting HA with hyaluronidase, suggesting that HA is only partially responsible for tribological behavior of the joint. Loss of lubricin results in the increased coefficient of friction of the joint, which may also be associated with increased wear, loss of range of motion, and pain.

Current conservative treatments for OA aim to mediate pain with analgesics, physical therapy and weight loss. In the end stage of arthritis, highly invasive surgical intervention is required, such as total knee arthroplasty, which results in 2-3 days of hospital time, 3-6 months of rehabilitation, 10% reduced range-of-motion and permanently compromised physical capabilities. The present working hypothesis is that viscosupplementation (i.e., increasing viscosity of the synovial fluid) provides long-term pain relief and increases mobility. Prior to total knee arthroplasty, viscosupplementation injections of hyaluronic acid (HA) (SYNVISC®, ORTHOVISC®, EUFLEXXA®) are also widely used in an attempt to restore joint lubrication and reduce cartilage erosion, however their effects last less than 6 months, with injected HA being enzymatically degraded in a joint in just 4 weeks. HA viscosupplementation market was valued at $1.4 billion in 2010, although the Academy of Orthopedic Surgeons in 2013 withdrew support for the procedure due to unreliable results, which fall at about 50% success for pain relief and recovery of range-of-motion.

OA affects more than 20 million people in the U.S., making it the leading cause of chronic disability in people over the age of 70 and costing 100 billion dollars annually. Incidence rates of osteoarthritis in active duty military personnel are significantly higher than in the general population of comparable age groups and to be highly age-dependent. For 35-39 y.o. age group incidence rate in military is as high as 14.21 versus 7.08 (per 1,000) for the general population. In addition, OA incidence rates for female military personnel were found to be 20% higher than that for their male colleagues.

There is thus a need in the art for novel effective, minimally-invasive OA treatment. The present invention fulfills this unmet need.

### BRIEF SUMMARY OF THE INVENTION

In certain aspects, the invention provides a method of treating or preventing osteoarthritis (OA) in a mammal in need thereof, the method comprising contacting an articulating joint of the mammal affected by OA with a therapeutically effective amount of a biomimetic proteoglycan.

In certain embodiments, the biomimetic proteoglycan is soluble in an aqueous solution. In other embodiments, the articulating joint is at least one selected from the group selected from a hand joint, wrist joint, shoulder joint, ankle joint, knee joint, hip joint, and spine joint.

In certain embodiments, the biomimetic proteoglycan comprises a core structure and at least one glycosaminoglycan (GAG), wherein the core structure and the GAG are covalently linked. In other embodiments, the GAG is at least one selected from the group consisting of hyaluronic acid, chondroitin, chondroitin sulfate, heparin, heparin sulfate, dermatan sulfate, laminin, keratin sulfate, chitin, chitosan, acetyl-glucosamine, and oligosaccharides. In other embodiments, the core structure is at least one selected from the group consisting of a synthetic polymer, a protein, a peptide, a nucleic acid, and a carbohydrate. In yet other embodiments, the synthetic polymer is at least one selected from the group consisting poly(4-vinylphenyl boronic acid), poly(3,3′-diethoxypropyl methacylate), polyacrolein, poly(N-isopropyl acrylamide-co-glycidyl methacrylate), poly(allyl glycidyl ether), poly(ethylene glycol), poly(acrylic acid), poly(acryloyl chloride) and epoxides. In yet other embodiments, the core structure comprises polyacrylic acid (PAA).

In certain embodiments, the biomimetic proteoglycan comprises chondroitin sulfate and PAA, wherein the chondroitin sulfate is covalently linked to the PAA.

In certain embodiments, the biomimetic proteoglycan is resistant to the breakdown of an endogenous enzyme. In other embodiments, the endogenous enzyme is at least one selected from the group consisting of hyaluronidases, aggrecanases and matrix metalloproteinases (MMPs).

In certain embodiments, the biomimetic proteoglycan is susceptible to the breakdown of an exogenous enzyme. In other embodiments, the exogenous enzyme is chondroitinase ABC (ChABC).

In certain embodiments, the GAG is attached to the core structure through a linkage selected from the group consisting of a boronic acid-diol linkage, epoxide-amine linkage, aldehyde-amine linkage, carboxylic acid-amine linkage, sulfhydryl-maleimide linkage, and any combinations thereof. In other embodiments, the GAG comprises at least one terminal handle selected from the group consisting of a primary amine, diol, and aldehyde.

In certain embodiments, the biomimetic proteoglycan comprises a synthetic polymer, wherein the backbone of the synthetic polymer is attached to at least one sulfated glycosaminoglycan (GAG) through a terminal amino group of the at least one sulfated GAG wherein the synthetic polymer is soluble in an aqueous solution. In other embodiments, the at least one sulfated GAG is selected from the group consisting of chondroitin sulfate, heparin sulfate, dermatin sulfate, keratin sulfate, and any combinations thereof. In yet other embodiments, the synthetic polymeric backbone is at least one selected from the group consisting poly(4-vinylphenyl boronic acid), poly(3,3′-diethoxypropyl methacylate), polyacrolein, poly(N-isopropyl acrylamide-coglycidyl methacrylate), poly(allyl glycidyl ether), poly(ethylene glycol), and poly(acrylic acid). In yet other embodiments, the at least one sulfated GAG is attached to the synthetic polymer backbone by way of a linking chemistry selected from the group consisting of epoxide-amine linkage, aldehyde-amine linkage, carboxylic acid-amine, acyl chloride amine linkage, and any combinations thereof.

In certain embodiments, the biomimetic proteoglycan has at least one shape selected from the group consisting of cyclic, linear, branched, star-shaped, comb, graft, bottlebrush, dendritic, and mushroom.

In certain embodiments, the biomimetic proteoglycan has at least one property that mimics a natural sulfated proteoglycan, wherein the at least one property is selected from the group consisting of water uptake and charge density.

In certain embodiments, at least a fraction of the biomimetic proteoglycan migrates into the cartilage that is in contact with the joint. In other embodiments, the fraction of the biomimetic proteoglycan that migrates into the cartilage localizes around the cells (chondrocytes) in the pericellular region or inter-territorial zone. In yet other embodiments, the biomimetic proteoglycan migrates to the extracellular matrix of the cartilage.

In certain embodiments, the biomimetic proteoglycan is crosslinked to itself and/or an additional molecule. In other embodiments, the additional molecule is at least one selected from the group consisting of collagen, pectin, carrageenan, poly(L-lysine), gelatin, agarose, dextran sulfate, heparin, polygalacturonic acid, mucin, chondroitin sulfate, hyaluronic acid (HA), chitosan, alginate, alginate sulfate, poly(acrylic acid), poly(methyl methacrylate) (PMMA), poly(vinylsulfonic acid) (PVSA), poly(L-aspartic acid), poly(L-glutamic acid), poly(L-aspartic acid)-grafted-poly(ethylene glycol) (PAA-g-PEG), poly(L-glutamic acid)-grafted-poly(ethylene glycol) (PGA-g-PEG), poly(sodium 4-styrenesulfonate) (PSS), dermatan sulfate, and carboxymethyl cellulose (CMC).

In certain embodiments, the articulating joint is further contacted with a therapeutic agent that treats OA. In other embodiments, the biomimetic proteoglycan and therapeutic agent are co-administered to the mammal. In yet other embodiments, the biomimetic proteoglycan and therapeutic agent are co-formulated. In yet other embodiments, the therapeutic agent is at least one selected from the group consisting of acetaminophen, hyaluronic acid (HA), an anti-Fas antibody, a non-steroidal anti-inflammatory drug, a COX-2 inhibitor, a p21-activated kinase inhibitor, and an agent targeting promoting factors of cartilage reproduction.

In certain embodiments, the mammal is human.

The invention also provides a kit comprising a composition comprising at least one biomimetic proteoglycan, an applicator, and an instructional material for use thereof, wherein the instructional material comprises instructions for treating or preventing osteoarthritis in a mammal using the at least one biomimetic aggrecan.

### BRIEF DESCRIPTION OF THE DRAWINGS

The following detailed description of specific embodiments of the invention will be better understood when read in conjunction with the appended drawings. For the purpose of illustrating the invention, specific embodiments are illustrated in the drawings. It should be understood, however, that the invention is not limited to the precise arrangements and instrumentalities of the embodiments illustrated in the drawings.

FIG. 1A is a series of images illustrating that biomimetic proteoglycan (right) mimics 3D structure of natural aggrecan and infiltrates cartilage to modify the tissue (left). FIGS. 1B-1C are schematic representations of natural proteoglycans (FIG. 1B) and biomimetic proteoglycans (FIG. 1C). The exemplified biomimetic proteoglycans are fabricated by grafting chondroitin sulfate (CS) molecules onto a polymer core having an enzymatically resistant backbone. By varying core length and attachment density of chondroitin sulfate bristles, a family of biomimetic proteoglycans with different macromolecular architectures can be fabricated. CS—Chondroitin sulfate; KS—Keratin sulfate; DS—Dermatan sulfate; LRR—Leucine rich region.

FIG. 2 schematically depicts steps for synthesis of a biomimetic proteoglycan with polyacrylic acid (PAA) as the backbone and chondroitin sulfate (CS) as the bristles. The biomimetic proteoglycan forms a 3D bottle brush (a biomimetic proteoglycan or BPG).

FIG. 3A depicts the AFM images of natural aggrecans and PAA250-CS showing bottlebrush architecture. FIG. 3B depicts the AFM images of natural aggrecans and PAA10-CS showing short bottlebrush or star-shaped molecules. FIG. 3C is a graph showing the distribution of osmotic pressure at different concentrations of CS, PAA250-CS, and PAA10-CS. FIG. 3D is a bar graph illustrating water uptake capacity of different materials: aggrecan, CS, HA, PAA250-CS, and PAA10-CS. The BPGs show increased water uptake (˜60-63%) compared with CS (45%), natural aggrecan (43%) and 2,000 kDa hyaluronic acid (52%), which has been used as a viscosupplement for osteoarthritis treatment. FIG. 3E is a graph showing the rate of water uptake of aggrecan, CS, HA, PAA250-CS, and PAA10-CS. The BPGs show more rapid water uptake compared to CS, natural aggrecan and HA.

FIGS. 4A-4B are a series of live-dead images illustrating cytocompatibility of PAA250-CS (FIG. 4A) and PAA10-CS (FIG. 4B). FIG. 4C is a graph showing cell viability for cells exposed to PAA10-CS (BPG10) and CS.

FIG. 5A shows enzymatic digestion with hylyuronidase for biomimetic proteoglycans. FIG. 5B shows enzymatic digestion with ChABC and hyaluronidase for biomimetic proteoglycans.

FIGS. 6A-6C are a series of images illustrating fluorescently labeled biomimetic proteoglycans infiltrating cartilage.

FIG. 7 is a bar graph illustrating the area percent fluorescence from biomimetic proteoglycan infiltration. n=5 all groups. # denotes p<0.05 compared to control. * denotes p<0.05.

FIG. 8 is a set of confocal images of PAA10-CS diffusing into bovine articular cartilage for times up to 24 hours (scale bar 100 μm).

FIGS. 9A-9C are confocal images of cartilage samples (scale bar 100 μm). Contrast and fluorescent images for controls, CS, and PAA10-CS at Collins' grades 0-1 (FIG. 9B), 2-3 (FIG. 9B) and 4 (FIG. 9C). FIG. 9D is a patient data table reporting the source of the cartilage samples used in FIGS. 9A-9C.

FIG. 10 is a graph reporting the binding affinity of Natural Aggrecan (AG), Biomimetic Proteoglycan (BA), and Chondroitin Sulfate (CS) to Collagen II.

FIG. 11A is a representative schematic of a custom built pendulum for measuring whole joint coefficient of friction for rabbit joints. FIG. 11B is a representative “angle vs time” pendulum output showing angular decay over time.

FIGS. 12A-12E are images of articular cartilage samples from the tibial plateau stained with Safranin-O: (FIG. 12A) Normal, (FIG. 12B) PAA10-CS only, (FIG. 12C) OA control, (FIG. 12D) OA+PAA10-CS injection, (FIG. 12E) OA+HA.

FIGS. 13A-13E are a series of confocal microscopy images illustrating the diffusion of PAA10-CS into the cartilage matrix after being injected into the rabbit knee joint in vivo. FIGS. 13A-13B are images of a normal, non-injected rabbit. FIGS. 13C-13D are images of a rabbit injected with fluorescently labeled PAA10CS. FIG. 13E is an enlarged area of fluorescently injected rabbit cartilage which removes a folding artifact.

FIG. 14 is a graph showing the coefficient of friction results obtained from the pendulum friction test. Points represent the mean±standard deviation for each experimental group. P=0.033 for Normal vs OA. P=0.049 for Normal vs OA+HA.

### DETAILED DESCRIPTION OF THE INVENTION

The present invention relates to novel methods of treating or preventing arthritis for a mammal in need thereof. In certain embodiments, the method comprises contacting a biomimetic proteoglycan with the affected joints of the mammal. In other embodiments, the method treats osteoarthritis (OA). In other embodiments, the OA comprises knee osteoarthritis, such as but not limited to post-traumatic knee osteoarthritis. The methods of the invention are in certain embodiments minimally invasive, alleviate pain, reduce tissue degeneration, and/or have long-lasting effect in the mammal.

As demonstrated herein, a highly hydrating, but not highly viscous, injection to the joint space increases joint lubrication, challenging the existing paradigm of OA treatment using viscosupplementation. Without wishing to be limited by any theory, in order to restore pain relief and increase mobility of the knee, a low friction interface is restored to the joint. Injections of biomimetic proteoglycan into the joint space allow for a two-pronged approach to the treatment of the arthritic knee: 1) reducing friction of the joint by increasing hydration and restoring a boundary lubrication mechanism, and 2) restoring the cartilage extracellular matrix by allowing the migration of biomimetic proteoglycan from the joint intra-articular space into the cartilage extracellular matrix, where it integrates with existing collagens in the extracellular matrix or pericellular matrix or interterritorial zone. Without being limited to any particular theory, this integration creates increased hydration and osmotic pressure and restores more normal mechanical properties to the tissue. Injection of biomimetic proteoglycan into the knee joint (intra-articular space) is a novel minimally invasive OA clinical treatment for the general population.

As demonstrated herein, biomimetic proteoglycans (BPGs) were designed to mimic the 3D bottle brush architecture of natural aggrecan. The BPGs have physical structure and properties (hydration and water uptake) that mimic that of natural aggrecan. The BPGs can infiltrate cartilage to molecularly engineer the tissue and have the potential to affect mechanical behavior of cartilage. The BPGs are cytocompatible with no adverse effect on proliferation. The BPGs have a similar binding affinity to collagen II as natural aggrecan, which has potential to aid in retention of the BPGs in cartilage tissue.

### Definitions

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

The term “about” will be understood by persons of ordinary skill in the art and will vary to some extent on the context in which it is used. “About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

As used herein, the term “ADAMT” refers to A Disintegrin And Metalloproteinase with Thrombospondin Motif.

As used herein, the term “aggrecan” refers to the aggrecan monomer.

As used herein, the term “aggregated aggrecan” refers to aggrecan that is linked to hyaluronic acid in an end-on configuration and comprises about 100 aggrecan monomers.

As used herein, a disease or disorder is “alleviated” if the severity of a symptom of the disease or disorder, the frequency with which such a symptom is experienced by a patient, or both, is reduced.

As used herein, the terms “BA” and “BPG” both refer to biomimetic proteoglycan.

The term “biocompatible polymer” refers to a polymer that is non-toxic, chemically inert, non-irritable, insoluble and substantially non-immunogenic when contacted with human tissue.

The term “biologically compatible” or “biocompatible” refers to reagents, cells, compounds, materials, compositions, and/or dosage formulations that are suitable for use in contact with the tissues of human beings and animals without excessive toxicity, irritation, allergic response, or other complication commensurate with a reasonable benefit/risk ratio.

As used herein, the term “biomimetic proteoglycan” refers to a material that mimics at least certain aspects of the natural structure and function of natural aggrecan, such as, but not limited to, hydration, structure support, and interaction with the existing tissue including joint cartilage.

As used herein, the term “bristle” refers to the glycosaminoglycan chain covalently connected a core structure of the invention.

The terms “core structure” and “polymer backbone,” as used interchangeably herein, refer to the moiety or structure for which GAGs, such as chondroitin sulfate, can attach to form a biomimetic proteoglycan. In some instances, the core structure is considered the polymer backbone, core portion, polymer core, or protein portion of the aggrecan. In some instances, the core structure can be a synthetic polymer, protein, peptide, nucleic acid, carbohydrate or combinations thereof.

As used herein, the term “CS” refers to chondroitin sulfate.

As used herein, the term “Da” refers to the unified atomic mass unit or Dalton, which is the standard unit of indicating mass of an atom or a molecular.

As used herein, a “disease” is a state of health of an animal wherein the animal cannot maintain homeostasis, and wherein if the disease is not ameliorated then the animal's health continues to deteriorate.

As used herein, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, the term “ECM” refers to extracellular matrix.

As used herein, the term “endogenous enzyme” refers to the enzyme that originates from the particular tissue of a subject.

As used herein, the term “exogenous enzyme” refers to the enzyme that does not originate from the particular tissue of a subject.

The terms “glycosaminoglycan” and “GAG”, as used interchangeably herein, refer to a macromolecule comprised of carbohydrate. The GAGs for use in the present invention can vary in size and be either sulfated or non-sulfated. The GAGs that are contemplated within the invention include, but are not limited to, hyaluronic acid, chondroitin, chondroitin sulfates (e.g., chondroitin 6-sulfate and chondroitin 4-sulfate), heparin, heparin sulfate, dermatan sulfate, laminin, keratan sulfate, chitin, chitosan, acetyl-glucosamine, and the like.

As used herein, “grafting from” refers to a process based on the synthesis of a macroinitiator containing suitable initiating groups along the backbone. The high initiator efficiency, the ability to manipulate initiator distribution along the main chain and the side chain length control afforded by living polymerization techniques makes the “grafting from” process an attractive option in the synthesis of well-defined graft copolymers.

As used herein, “grafting through” refers to a process of the synthesis of a well-defined macromonomer, followed by its copolymerization with a low molecular weight co-monomer. Control over length and polydispersity can be achieved for both backbone and side chains using this methodology. The approach is characteristic of a multistep synthesis and the grafting density is associated with the reactivity ratios of the macromonomers.

As used herein, the term “grafting to” or “grafting onto” refers to end-functionalized polymer chains that are attached to the main chain of another polymer through coupling reactions with one or more functional groups along its backbone.

As used herein, the term “HA” refers to hyaluronic acid.

As used herein, an “instructional material” includes a publication, a recording, a diagram, or any other medium of expression that can be used to communicate the usefulness of a composition or method of the invention in the kit for treating, preventing or alleviating various diseases or disorders recited herein. The instructional material of the kit of the invention can, for example, be affixed to a container that contains the identified composition or delivery system of the invention or be shipped together with a container that contains the identified composition or delivery system. Alternatively, the instructional material can be shipped separately from the container with the intention that the instructional material and the composition be used cooperatively by the recipient.

As used herein, the term “MMP” refers to matrix metalloproteinase.

As used herein, the term “OA” refers to osteoarthritis.

As used herein, the terms “patient,” “subject,” “individual” and the like are used interchangeably, and refer to any animal, or organs, tissues or cells thereof whether in vitro or in situ, amenable to the methods described herein. In certain embodiments, the patient, subject or individual is a vertebrate. In other embodiments, the patient, subject or individual is a mammal. Non-human mammals include, for example, livestock and pets, such as ovine, bovine, porcine, canine, feline, equine and murine mammals. In yet other embodiments, the patient, subject or individual is a human.

As used herein, the term “PAA10-CS” refers to a biomimetic proteoglycan synthesized from polylactic acid with a molecular weight of 10 kDa and chondroitin sulfate.

As used herein, the term “PAA250-CS” refers to a biomimetic proteoglycan synthesized from polylactic acid with a molecular weight of 250 kDa and chondroitin sulfate.

As used herein, “terminal handle” refers to the functional group at the end of GAG, through which the GAG is attached to the core structure.

As used herein, the term “therapeutically effective amount” refers to an amount that is sufficient or effective to prevent or treat (delay or prevent the onset of, prevent the progression of, inhibit, decrease or reverse) a disease or condition described or contemplated herein, including alleviating symptoms of such disease or condition.

As used herein, the term “treating a disease or disorder” means reducing the frequency with which a symptom of the disease or disorder is experienced by a patient. Disease and disorder are used interchangeably herein.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 and the like, as well as individual numbers within that range, for example, 1, 2, 2.7, 3, 4, 5, 5.3, and 6. This applies regardless of the breadth of the range.

### DISCLOSURE

The present invention relates to novel methods of treating or preventing OA for a mammal in need thereof. In one aspect, the new method comprises contacting a biomimetic proteoglycan, which is optionally formulated as a pharmaceutically acceptable composition, with a joint of a mammal affected by OA.

In another aspect, a biomimetic proteoglycan is used in combination with a therapeutic agent in the treatment of OA. Non-limiting examples of the therapeutic agents for treating OA include acetaminophen, an anti-Fas antibody, a non-steroidal anti-inflammatory drug, COX-2 inhibitor, a p21-activated kinase inhibitor, and an agent targeting promoting factors of cartilage reproduction such as interleukin (IL)-1. In certain embodiments, the biomimetic proteoglycan and the therapeutic agent are co-administered to the mammal. In other embodiments, the biomimetic proteoglycan and the therapeutic agent are co-formulated prior to the administration to the mammal.

The affected joint can be anywhere in the body of a mammal. In certain embodiments, the affected joint is at least one selected from the group selected from an articulating joint such as a hand joint, a wrist joint, a shoulder joint, an ankle joint, a knee joint, a hip joint, and a spine joint. In other embodiments, the spine joint is a facet joint. In yet other embodiments, the spine joint is an intervertebral disc and/or meniscus.

In one aspect, biomimetic proteoglycans mimic the structure and functionality of natural aggrecans. Natural aggrecans have a dense bottle-brush architecture with over 100 chondroitin sulfate (CS) bristles (as well as ˜30 keratin sulfate bristles) spaced 3-5 nm apart along the 250 kDa protein core with an overall molecular weight of ˜2,000 kDa. Biomimetic proteoglycans of the invention are designed and prepared to resist enzymatic cleavage. They are prepared by attaching a GAG chain via a covalent bond to a core structure. The covalent bond can be formed through, for example, boronic acid-diol reaction, epoxide-amine reaction, aldehyde-amine reaction, carboxylic acid-amine reaction, sulfhydryl-maleimide reaction, acyl chloride-amine and any combinations thereof. Examples of compositions comprising biomimetic proteoglycans are described in U.S. Application Publication No. US 2013/0052155 A1, which is hereby incorporated herein by reference in its entirety.

In certain embodiments, the biomimetic proteoglycans can be prepared by attaching a terminal diol in chondroitin sulfate to a boronic acid-functionalized polymer. Utilizing the high affinity complexation of boronic acids with compounds containing diols (such as saccharides), novel biomimetic proteoglycans may be prepared, wherein the attached chondroitin sulfate chains form brush “bristles” to mimic the bristles of the natural aggrecan molecule.

In other embodiments, the biomimetic proteoglycans can be prepared by attaching at least one chondroitin sulfate through a terminal primary amine handle to a diverse array of polymer backbones. A primary amine in the terminal region of the chondroitin sulfate molecule allows for the controlled organization of chondroitin sulfate onto various polymeric backbones.

In yet other embodiments, the biomimetic proteoglycans can be prepared using a grafting method wherein chondroitin sulfate or other similar GAG chain is grafted to a functional polymer. The functional polymer can be, but is not limited to, any polymer with groups that may react with diols or primary amines, such as boronic acids, epoxides, aldehydes and/or carboxylic acids. An example of a possible “grafted to” polymer is poly(acrylic acid), which is a carboxylic acid-containing linear polymer. Poly(acrylic acid) may be activated with 1-ethyl-3-(3-dimethyl aminopropyl) carbodiimide hydrochloride (EDC)/N-hydroxysulfosuccinimide (NETS) and then reacted with the terminal primary amine of chondroitin sulfate thus creating a bottle brush structure.

In yet other embodiments, the biomimetic proteoglycans are prepared through the “grafting through” method, wherein the chondroitin sulfate or other similar GAG chain is modified with a polymerizable end group which is subsequently homo- or co-polymerized to form a bottle brush polymer. As an example of a possible “grafted through” polymer, 2-vinyl oxirane may be attached to chondroitin sulfate through an interaction of the terminal primary amine in chondroitin sulfate with the epoxide of 2-vinyloxirane, thus creating a vinyl chondroitin sulfate. The vinyl chondroitin sulfate may be subsequently polymerized via free radical polymerization. Similarly, another example is the attachment of poly(4-vinylbenzylboronic acid) to chondroitin sulfate via an interaction of the terminal diol in chondroitin sulfate with the boronic acid in poly(4-vinylbenzylboronic acid) to form a boronic ester. The vinyl-containing chondroitin sulfate is then subsequently polymerized via free radical polymerization to form a bottle brush polymer.

In yet other embodiments, the biomimetic proteoglycans are prepared through the grafting method, wherein a disaccharide unit of chondroitin sulfate (GlcUA and GalNAc) or other GAG is attached to a polymeric backbone, through but not limited to aldehyde or amine interactions. Subsequent disaccharide or saccharide units are then grown from the polymeric backbone using enzymes of GAG synthesis, such as but not limited to GlcA I transferase, GlaNAc transferase, chondroitin synthase, chondroitin 6-sulfotransferase and chondroitin 4-O-sulfotransferase.

In yet other embodiments, the biomimetic proteoglycans prepared through any of the grafting methods are end-functionalized with, but not limited to, a hyaluronic binding region or collagen binding region. Polymerizations that can be utilized to incorporate a functional group on the terminal end of the biomimetic proteoglycans bottle brush include, but are not limited to, radical polymerization, cationic polymerization, living anionic polymerization, atom transfer radical polymerization, and ring opening metathesis polymerization.

Based on the disclosure herein, a skilled artisan would understand that the biomimetic proteoglycans can be engineered to encompass any type of glycosaminoglycan and combinations thereof with any type of core protein or polymer.

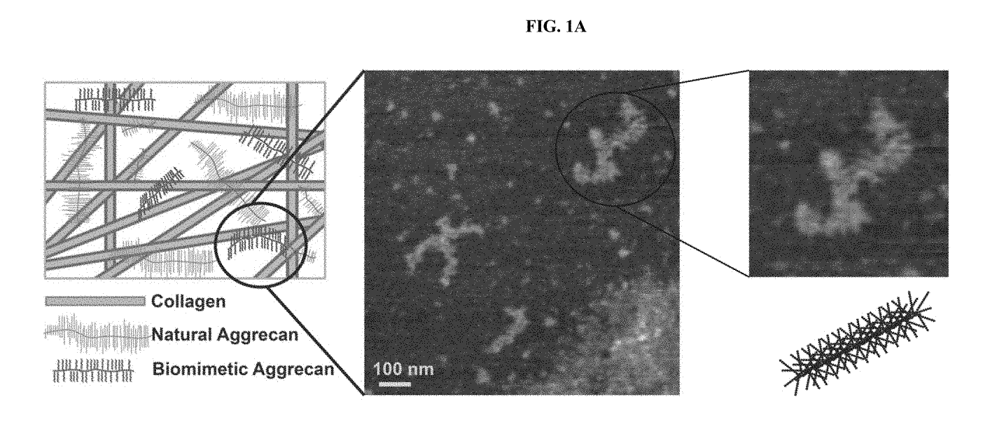

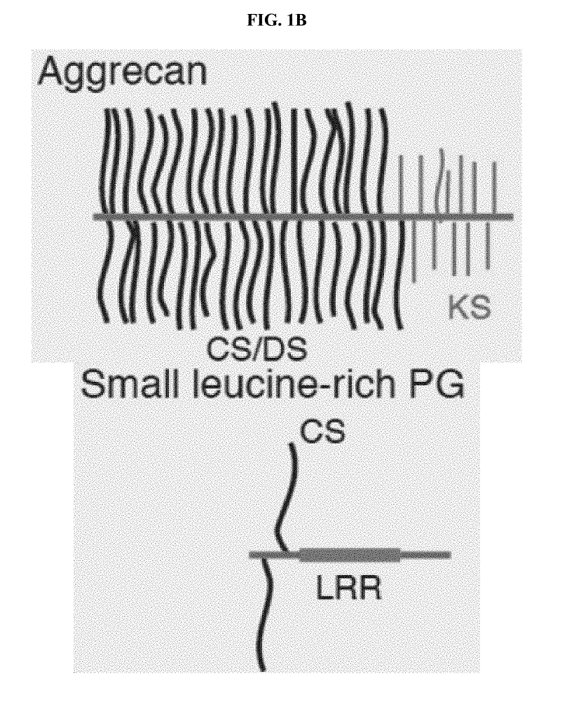

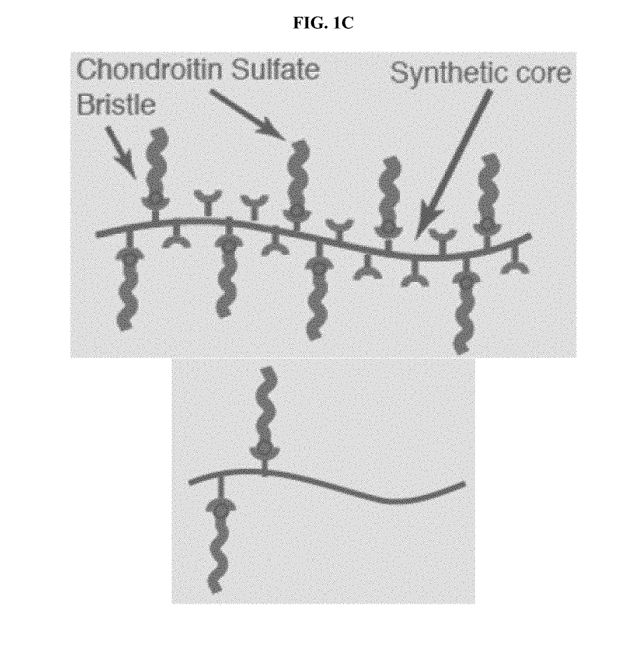

In certain embodiments, the biomimetic proteoglycan comprises natural chondroitin sulfate bristles attached to an enzymatically resistant synthetic polymer backbone poly(acrylic acid). In other embodiments, the biomimetic proteoglycan mimics the 3D bottle-brush architecture (FIGS. 1A-1C) and osmotic and water uptake properties of the natural proteoglycans, such as aggrecan, critical to the function of cartilage. In yet other embodiments, the biomimetic proteoglycan is cytocompatible, has similar binding affinity to collagen II as natural aggrecan, and is resistant to the native intra-articular enzyme hyaluronidase that breaks down all current HA injection treatments. Without wishing to be limited by any theory, this may be due to the biocompatible synthetic polymer backbone, which replaces the enzymatically-sensitive protein core of the natural aggrecan molecule. In certain embodiments, the biomimetic proteoglycan lubricates the joint surface, hydrates the OA cartilage, provides structural properties to arthritic cartilage through its 3D architecture and remains in tissue for a longer time than currently used HA, due to its enzymatic resistance and integration with existing collagen in the ECM.

Although natural aggrecan monomer exists in bottlebrush shape, it is understood that biomimetic proteoglycans, by design, may have a shape selected from the group consisting of cyclic, linear, branched, star-shaped, comb, graft, bottlebrush, dendritic, mushroom, and any combinations thereof. In certain embodiments, the biomimetic proteoglycans are star-shaped or bottlebrush shaped.

In certain embodiments, when biomimetic proteoglycans are arranged in a bottle-brush structure, such that the electrostatically charged bristle molecules are in close proximity to one another, the charged bristles provide electrostatic repulsions (by virtue of charge density distribution) and steric hindrances that assist the biomimetic proteoglycans in resisting force. This allows for an increased osmotic potential as well as improved mechanical function. Higher osmotic potential leads to greater retention of water by the polymer from the surroundings and stronger bulking effect.

In certain embodiments, the density of the bristles in the biomimetic proteoglycans is controllable, based on the careful selection of starting materials for the preparation of the biomimetic proteoglycans, whereby the spacing among the bristles can be varied to afford compositions of desired properties. In one embodiment, the bristle density is such that spacing among the bristles ranges from about 1 nm to about 12 nm, about 2 nm to about 9 nm, about 3 nm to about 8 nm, or about 4 nm to about 7 nm.

It will be understood that all kinds of glycosaminoglycans can be used to form a biomimetic proteoglycan. For example, suitable glycosaminoglycans include HA, chondroitin, chondroitin sulfate, dermatan sulfate, heparan sulfate, keratin sulfate and heparin. In addition, any polymer that resembles a glycosaminoglycan can be used to generate the biomimetic proteoglycans. Based on the disclosure presented herein, a skilled artisan would understand that any hydrophilic polymer can be used within the compositions of the invention.

In certain embodiments, the core structure is selected from the group consisting of a synthetic polymer, a protein, a peptide, a nucleic acid, a carbohydrate and any combinations thereof. In other embodiments, the core structure is a synthetic polymer selected from the group consisting poly(4-vinylphenyl boronic acid), poly(3,3′-diethoxypropyl methacrylate), polyacrolein, poly(N-isopropyl acrylamide-co-glycidyl methacrylate), poly(allyl glycidyl ether), poly(ethylene glycol), poly(acrylic acid), epoxides, poly(acryloyl chloride) and any combinations thereof. In certain embodiments, the biomimetic proteoglycans comprises a bottle brush-like molecule with CS bristles and polyacrylic acid as the polymer backbone. In other embodiments, the biomimetic proteoglycans comprises a bottle brush-like molecule with keratin sulfate bristles and polyacrylic acid as the polymer backbone. In other embodiments, the size of the biomimetic proteoglycans can be controlled so that a desired size is generated. In certain embodiments, the mean hydrodynamic size of the biomimetic proteoglycans is about 25 nm to about 1,000 nm, about 100 nm to about 300 nm, about 125 nm to about 275 nm, about 150 nm to about 250 nm, or about 180 nm to about 250 nm, or about 300 nm to about 1,000 nm.

In certain embodiments, the biomimetic proteoglycans are resistant to endogenous enzymatic digestion, so that the composition does not undergo enzymatic breakdown over a period of time, allowing for the repeated introduction of distinct components of the biomimetic proteoglycans onto an existing structure. Therefore, large macromolecular sized biomimetic proteoglycans can be generated over time. In certain embodiments, the endogenous enzyme is selected from the group consisting of hyaluronidases, aggrecanases and MMPs. In certain embodiments, the biomimetic proteoglycan is susceptible to exogenous enzymatic digestion. Specific breakdown of the molecule by exogenous enzymes may be used as a method to reverse the effects of introducing biomimetic proteoglycans into the subject. In one embodiment, the chondroitin sulfate bristles are degraded using exogenous chondroitinase, such as bacterially derived chondroitinase, such as chondroitinase ABC.

In certain embodiments, the biomimetic proteoglycans of the invention comprise a synthetic polymer, wherein the backbone of the synthetic polymer is attached to at least one sulfated GAG through a terminal amino group of the at least one sulfated GAG wherein the synthetic polymer is soluble in an aqueous solution; and wherein the synthetic polymer has at least one property that mimics a natural sulfated proteoglycan, wherein the at least one property is selected from the group consisting of water uptake and charge density. In other embodiments, the at least one sulfated GAG is selected from the group consisting of chondroitin sulfate, heparin sulfate, dermatin sulfate, keratan sulfate, and any combinations thereof. In yet other embodiments, at least one synthetic polymer property selected from the group consisting of water uptake and charge density is modified by varying the number of sulfated GAGs attached to the synthetic polymeric backbone. In yet other embodiments, the synthetic polymeric backbone is selected from the group consisting poly(4-vinylphenyl boronic acid), poly(3,3′-diethoxypropyl methacylate), polyacrolein, poly(N-isopropyl acrylamide-coglycidyl methacrylate), poly(allyl glycidyl ether), poly(ethylene glycol), poly(acrylic acid), and any combinations thereof. In yet other embodiments, the synthetic polymeric backbone renders the synthetic polymer resistant to enzymatic breakdown in a mammalian in vivo environment. In yet other embodiments, the at least one sulfated GAG is attached to the synthetic polymer backbone by way of a linking chemistry selected from the group consisting of epoxide-amine linkage, aldehyde-amine linkage, carboxylic acid-amine linkage, and any combinations.

In certain embodiments, the biomimetic proteoglycans are injectable by a delivery device or can be formulated to be injectable. In one non-limiting example, the biomimetic proteoglycan of the invention is injected to the knee joint intra-articular space. The biomimetic proteoglycan migrates into the cartilage after injection from the joint space and resides in the joint for at least five days. By comparison, non-crosslinked HA concentration has been shown to rapidly diminish after intra-articular injection and only traces of HA are present in articular cartilage and synovial fluid 2 days post-injection (Jackson, et al., Osteoarthritis and Cartilage, 2005(14):1248-1257). While cross-linked HA has a longer residence time (at least up to 28 days), it was only observed as globules floating in synovial fluid or adhered to articular cartilage surface. Further, the biomimetic proteoglycan localizes in the pericellular or inter-territorial zone. Physically, the incorporation of biomimetic proteoglycan into the cartilage tissue increases hydration and osmotic pressure which are partially responsible for joint lubrication. Without wishing to be limited to any one theory, this increased joint lubrication combats joint pain and degradation in arthritic subjects.

In certain embodiments, the biomimetic proteoglycans of the invention have the similar hydration capacity as HA (2 MDa), resists endogenous enzymatic degradation, and integrates with the existing cartilage tissue matrix (FIG. 1A). The molecular weight the biomimetic proteoglycans of the invention ranges from about 25 kDa to about 3,000 kDa, about 200 kDa to about 2,700 kDa, about 300 kDa to about 2,500 kDa, about 400 kDa to about 2,250 kDa, about 500 kDa to about 2,000 kDa, or about 600 kDa to about 1,500 kDa.

In certain embodiments, the biomimetic proteoglycan of the invention is crosslinked to itself and/or an additional molecule. The additional molecule is selected from the group consisting of collagen, pectin, carrageenan, poly(L-lysine), gelatin, agarose, dextran sulfate, heparin, polygalacturonic acid, mucin, chondroitin sulfate, hyaluronic acid (HA), chitosan, alginate, alginate sulfate, poly(acrylic acid), poly(methyl methacrylate) (PMMA), poly(vinylsulfonic acid) (PVSA), poly(L-aspartic acid), poly(L-glutamic acid), poly(L-aspartic acid)-grafted-poly(ethylene glycol) (PAA-g-PEG), poly(L-glutamic acid)-grafted-poly(ethylene glycol) (PGA-g-PEG), poly(sodium 4-styrenesulfonate) (PSS), dermatan sulfate, carboxymethyl cellulose (CMC), and any combinations thereof.

The invention further provides a kit for treating or preventing osteoarthritis. In certain embodiments, the kit comprises a composition, an applicator, and a delivery device, wherein the composition comprises at least one biomimetic proteoglycan.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present invention. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth herein.

### EXPERIMENTAL EXAMPLES

The invention is now described with reference to the following Examples. These Examples are provided for the purpose of illustration only, and the invention is not limited to these Examples, but rather encompasses all variations that are evident as a result of the teachings provided herein.

### Example 1. Synthesis of Biomimetic Proteoglycan

Synthesis of biomimetic proteoglycan can be achieved, for example, by reacting the terminal portion of CS with an appropriate group on the core structure to form a covalent bond. The chemistry link used for such attachment may comprise, for example, amine-epoxy, diol-boronic acid and so forth.

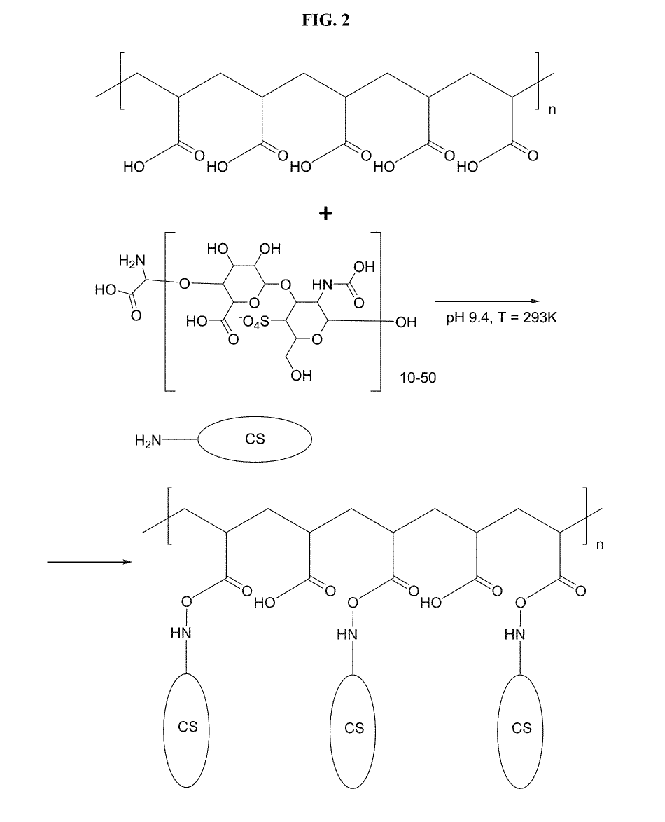

BPG was synthesized using a grafting-to strategy where CS bristles were assembled to a PAA core (FIG. 2). PAA is a linear polymer with an enzymatically resistant hydrocarbon backbone and pendant carboxylic acid groups. A functional primary amine (PA) was identified on the terminal end of chondroitin sulfate (CS-4, Sigma-Aldrich, MW ˜22 kDa). The PA functional group was used to incorporate CS into a macromolecule by grafting covalently to carboxylic acid functional groups on a linear, long chain polymer like PAA (“grafting-to”). Two different reaction strategies were used for the synthesis: the carboxylic acids of PAA were activated via reaction with EDC/sulfo-NHS for further reaction with the primary amine of CS (PBS, pH 7.4); and PAA10-CS was prepared by grafting CS onto poly(acryloyl chloride) (PAC) backbone (MW 10 kDa) via reaction between the terminal primary amine on CS and acyl chloride groups on PAC. Unreacted acyl chlorides are further hydrolyzed during synthesis into carboxyl groups resulting in a PAA backbone. Reaction between acyl chlorides on PAC and CS proceeded without additional activation via interface polymerization.

Large bottle brush molecules using 250 kDa MW PAA core with 1:15 CS:AA mol ratio (PAA250-CS) and 10 kDa MW PAC core with 1:10 CS:AC mol ratio (PAA10-CS) were synthesized. In certain embodiments, for synthesis of PAA250-CS, PAA (33 mg/mL, 0.132 mM PAA) was first activated with EDC/sulfo-NHS (2 mM EDC in DMSO and 5 mM sulfo-NHS in MES buffer (0.05M MES, 0.5M NaCl); pH 6.0) for 15-20 min followed by filtering using a Sephadex G-25 pre-packed desalting column (PD-10, GE Healthcare) to remove excess reactants. The column was centrifuged at 1500 G for 2 min. Activated PAA was then combined with a CS solution prepared in PBS. Solution pH was adjusted with NaOH to pH 7.4 and the mixture was allowed to react over time mixed continuously (THERMOLYNE LABQUAKE™) After 72 hours of reaction, samples were dialyzed in 50,000 Molecular Weight Cut Off (MWCO) membranes (Spectrum Labs, Rancho Dominguez, Calif.) against Dl water for 96 hours, then frozen and lyophilized. Purified samples were reconstituted and used for further characterization.

### Example 2. Characterization of Biomimetic Proteoglycan with PAA as the Backbone

Conjugation between CS and polymer backbones was successfully achieved and confirmed with a fluorescamine assay. Unreacted CS and monomers were removed by extensive dialysis (96 h against 1.5 L DI water), and the final polymer product was lyophilized. Chemical structures of the biomimetic proteoglycans were confirmed via ¹H NMR (300 MHz UNITYINOVA™ NMR Spectrometer, 30 mg/mL samples in D₂O) showing characteristic peaks for CS and corresponding polymer backbones for all purified compounds. After synthesis and purification, no degradation of CS bristles was observed. No traces of EDC or NHS were observed by NMR after dialysis; therefore any potential residuals that may remain in the molecules were below the detection limit of NMR. At a concentration of 300 mg/mL PAA250-CS or PAA10-CS solutions, the highest concentrations examined, no gelation was observed. This result indicates that at clinically relevant injection concentrations the molecules stay in solution, thus enabling them to integrate with the existing degenerative tissue.

### Dynamic Light Scattering

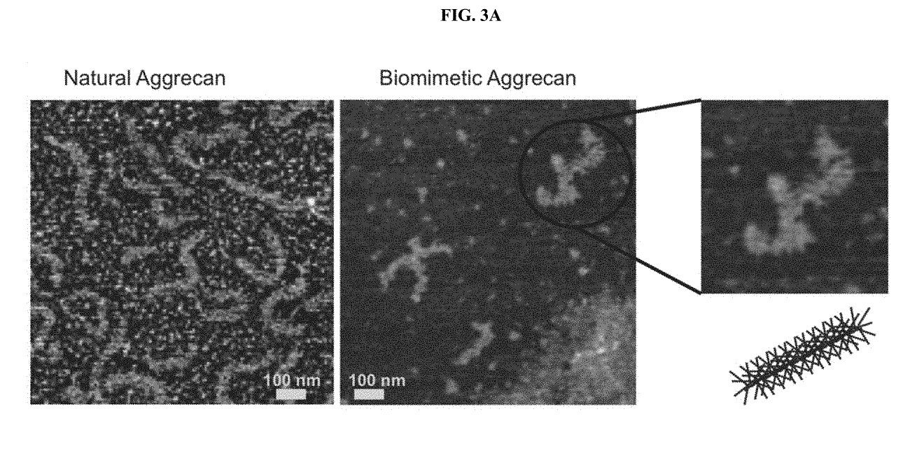

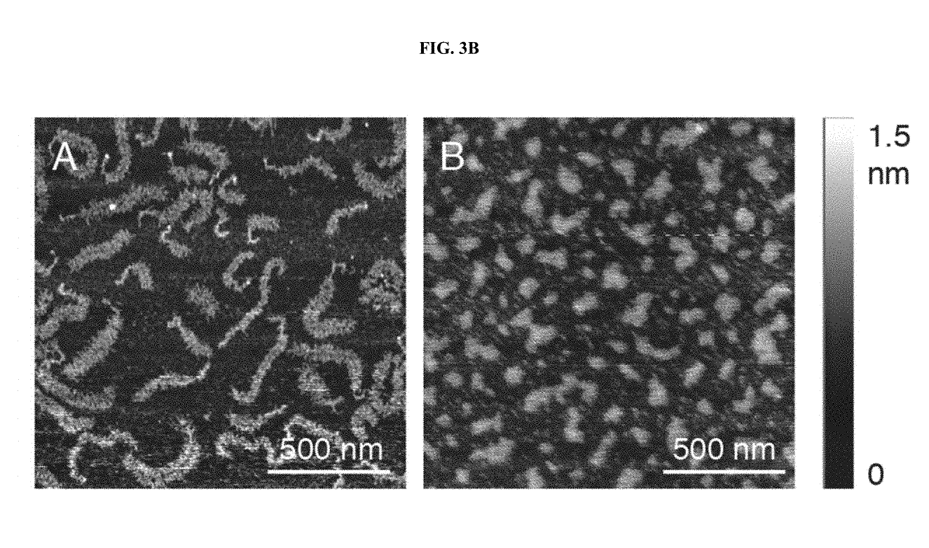

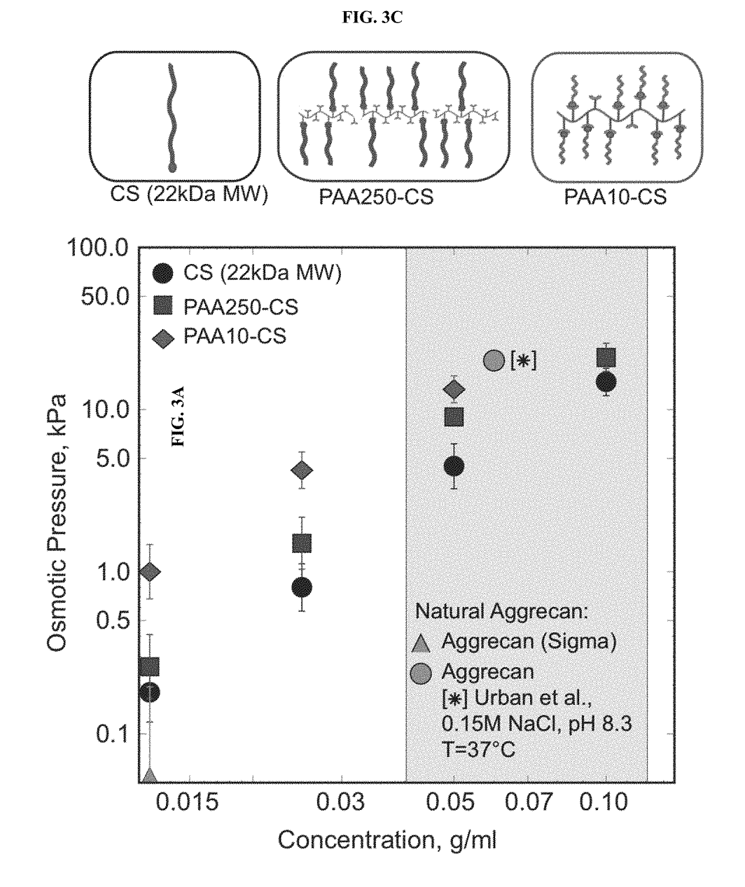

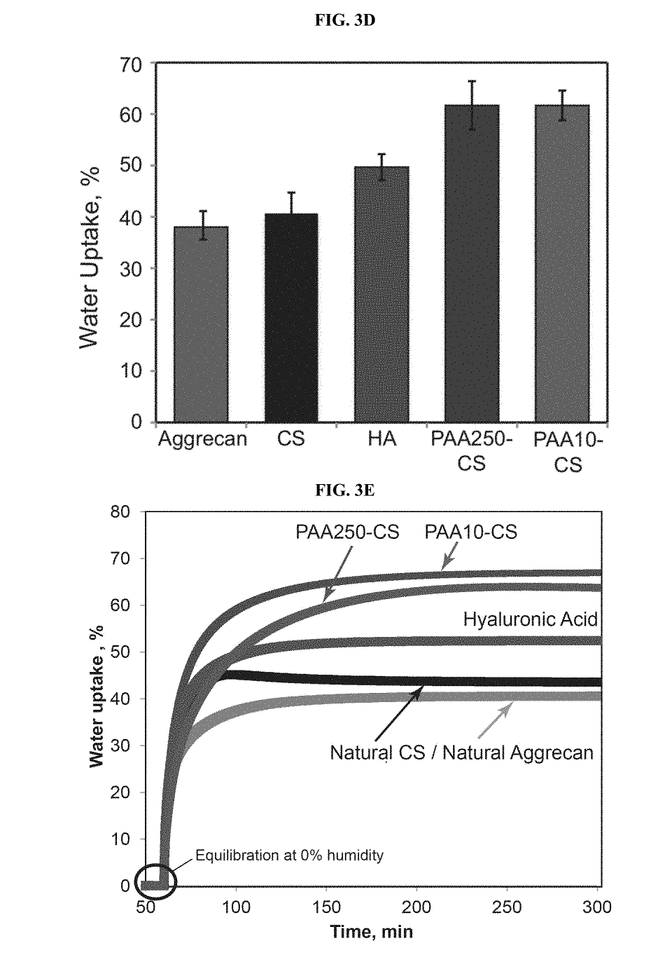

Atomic force microscope (AFM) imaging (FIG. 3A) shows the length of PAA250-CS in the range of 160 nm, further confirming the approximate size of the biomimetic proteoglycan molecules in a bottle brush configuration. This compares to ˜100-300 nm for natural aggrecan. Theoretical estimation of the molecular weight of biomimetic proteoglycan samples was calculated based on conjugation data for CS, as determined using a fluorescamine assay, sensitive to the reduction in primary amines (PA) on CS. The calculations suggested: PAA250-CS contains ˜60 CS bristles on the 250 kDa PAA core with bristles spaced at 14 nm and estimated MW of ˜1,600 kDa. In addition, PAA10-CS atomic force microscopy results (FIG. 3B) show that the molecules resemble star-shaped or short bottlebrush shapes containing ˜7-8 CS bristles on the 10 kDa PAA core with bristles spaced at 3-5 nm and estimated MW of ˜160-180 kDa.

### Osmotic Pressure and Water Uptake

Osmotic pressure is important for cartilage because it provides the potential to draw water and hold contact pressures during articulation. Osmotic pressure of PAA250-CS, PAA10-CS and CS solutions was measured with Sephadex G-50 beads using gel osmometry (FIG. 3B). In this study, calibration was done with PEG solutions of known osmotic pressure. The experimental data show that the PAA250-CS and PAA10-CS are in the osmotic pressure range of natural aggrecan. Pilot water uptake measurements were performed on natural aggrecan, CS, PAA250-CS and PAA10-CS using thermogravimetric analysis at 90% relative humidity at 37° C. over 24 hrs after drying in a vacuum oven at 37° C. for three days (FIGS. 3C-3D). CS and natural aggrecan had comparable water uptake at 43% and 40%, respectively. Biomimetic proteoglycan PAA250-CS and PAA10-CS had water uptake of 60% and 63%, respectively. This finding demonstrated that biomimetic proteoglycan showed a 50% increase in water uptake compared to natural aggrecan or CS alone.

### Cytocompatibility

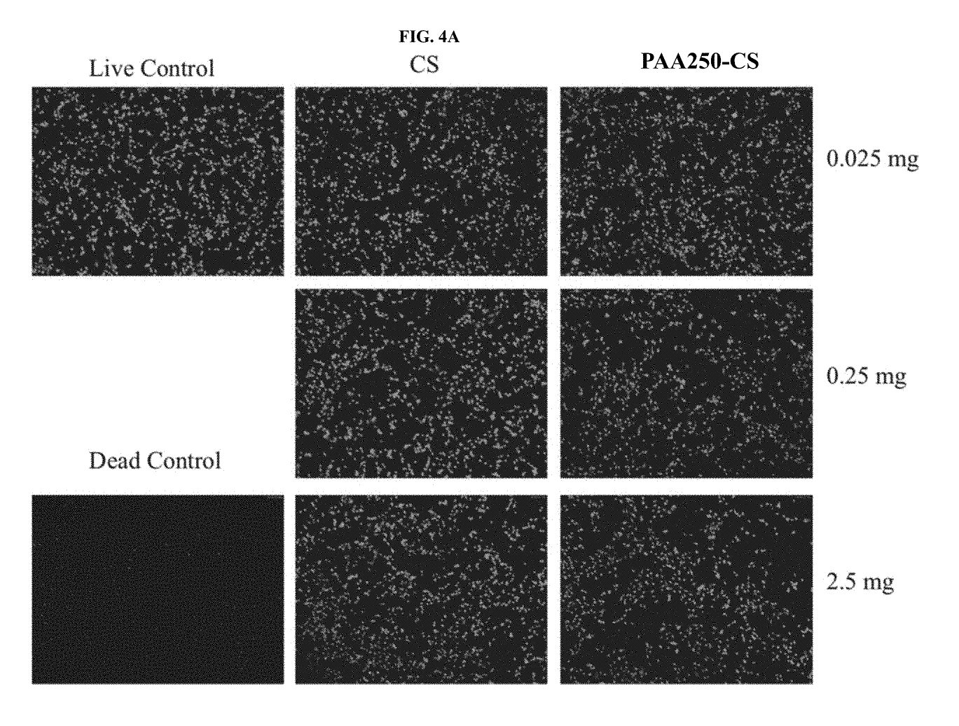

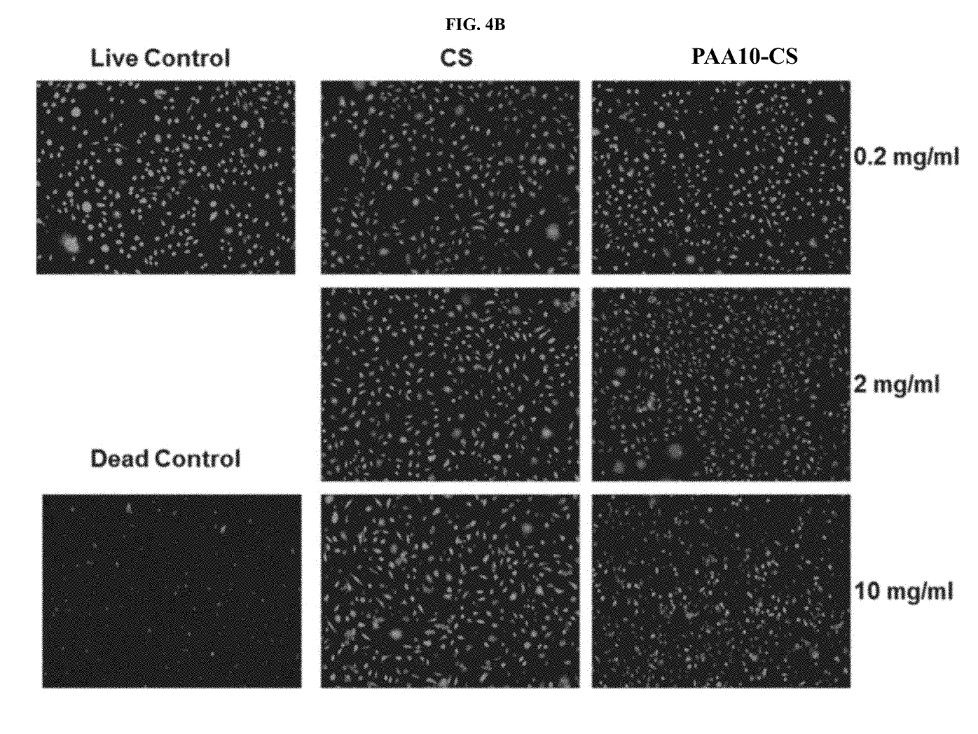

Cytocompatibility of PAA10-CS and PAA250-CS has been found to be concentration-dependent and comparable with CS at physiologically relevant concentrations (FIG. 4A-C). Cell viability study showed that PAA10-CS did not have an adverse effect on cell metabolic activity at concentrations of 0.02 and 0.2 mg/mL, as compared to natural CS (FIG. 4C). Further increase in PAA10-CS concentration to 2 mg/mL (˜4×10⁻⁶ mg of PG/cell) led to ˜20% decrease in the number of metabolically active cells, which could be caused by higher effective charge density of PAA10-CS due to additional ionic groups present on a polymer backbone. Similar trend was observed in cytotoxicity experiments, where, as indicated by Live/Dead Assay, both CS, PAA250-CS and PAA10-CS maintained cytocompatibility up to a concentration of 2.5 mg/mL (FIGS. 4A-4B). Both CS and PAA10-CS exhibit cytotoxicity at a concentration of 10 mg/mL. The range of 50-200×10⁶ cell/mL has been reported for cartilage (for <0.5 mm thickness). With ˜20-80 mg/mL concentration of PGs in cartilage, the natural PG/cell ratio can be estimated as 0.1-1.6×10⁻⁶ mg of PG/cell, which confirms that for high dosing concentration of BPG (such as 10 mg/mL in Live/Dead Assay), cells were subject to increased osmotic pressure and ionic conditions not common for native tissues.

### Enzymatic Digestion

The enzymatic stabilities of biomimetic proteoglycan and its major components (the synthetic polymer core and CS side-chains) were evaluated. These studies were observed using Blyscan, DMMB (1,9-dimethylmethylene blue).

First, reliability of the assays was confirmed by preparing a CS standard curve. The enzyme studies were designed with a positive and negative control. The enzyme degradation samples were heated to a biological temperature of 37° C. The change in absorption of samples with and without enzyme is an indication of GAG content, and therefore extent of degradation.

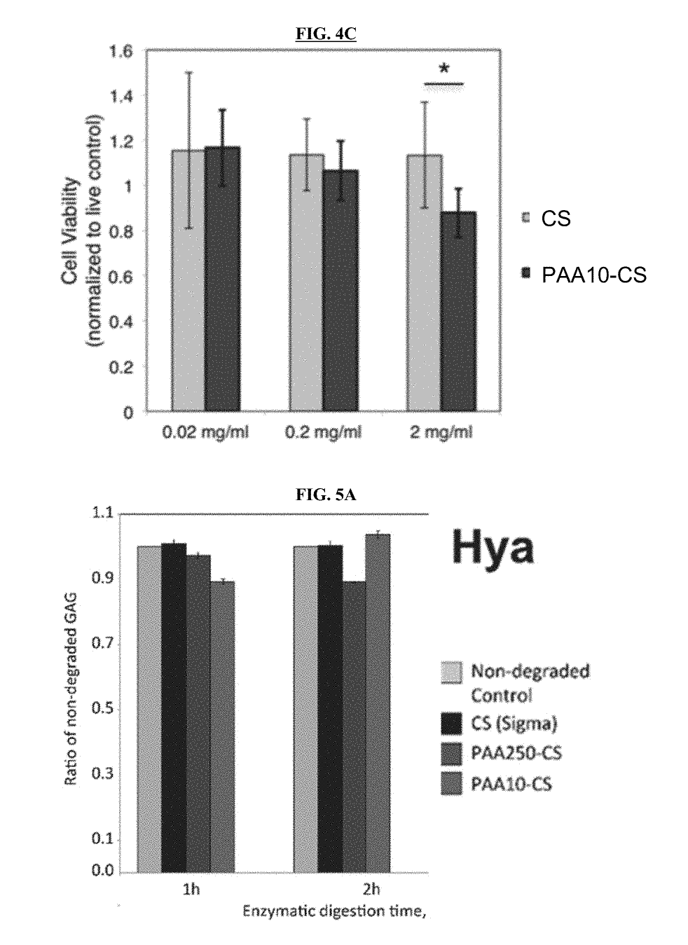

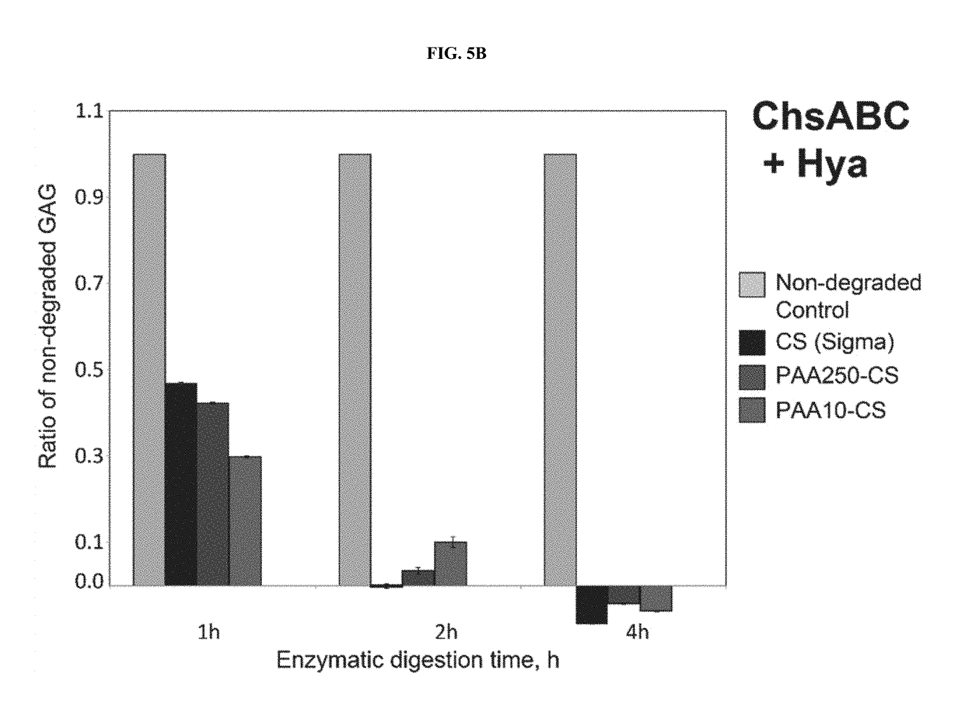

Biomimetic proteoglycan was incubated with mammalian hyaluronidase or hyaluronidase/exogenous chondroitinase ABC (ChABC) mixture. A concentrated solution was dissolved in a Tris/Acetate/Bovine Serum Albumin (BSA) buffer and diluted to 5 Units/ml. ChABC was reconstituted in 1 ml of 0.01% BSA. A 0.1 U/ml solution was used for enzymatic digestion studies. Biomimetic proteoglycan to be digested was prepared at 16 mg/mL of Tris/Acetate/BSA buffer. Samples were held for up 2 hrs at 37° C., then analyzed via DMMB or Blyscan to determine GAG concentrations. Hyaluronidase did not appreciably degrade biomimetic proteoglycan after being in solution for up to 2 hrs (FIG. 5A). However, when ChABC was added to hyaluronidase (positive control) there was significant degradation at 1 hr and complete degradation after 2 hrs (FIG. 5B). This promising finding suggests that, although biomimetic proteoglycan may be able to withstand the endogenous enzyme hyaluronidase (and also potentially aggrecanases and MMPs), if necessary, it may be enzymatically removed using exogenous ChABC.

### Example 3. Biomimetic Proteoglycan Infiltration and Distribution in Cartilage Ex Vivo

### In Vitro

Aggrecan is a vital component to the hydration and mechanical properties of articular cartilage and is lost during the early stages of OA. As demonstrated herein, the damaged cartilage is molecularly engineer with a novel biomimetic proteoglycan (BPG) to restore hydration and mechanical function. BPG mimics the 3-D bottle brush structure and properties of naturally occurring aggrecan and consists of a poly(acrylic acid) (PAA) core with CS bristles. The effect of BPG concentration, BPG molecular size, and media ionic strength on BPG infiltration into bovine osteochondral plugs was examined, and BPG distribution through the cartilage extracellular matrix (ECM) was evaluated.

Osteochondral plugs were cored from the femoral condyles of mature bovine knee joints. All BPG solutions were synthesized as in Example 1, fluorescently labeled with DCCH (7-Diethylaminocoumarin-3-Carboxylic acid, Hydrazide), and reconstituted in PBS. The osteochondral plugs were equilibrated in 1×PBS, then immersed in fluorescently labeled BPG solutions for 24 hours, with care taken only to expose only the articular surface to the solution. Plugs soaked in 1×PBS 10 mg/mL PAA10-CS (160-180 kDa) solution were used to compare all the BPG infiltration effects in this experiment. The remaining groups (n=5) were PAA250-CS (MW 1,600 kDa), 5 mg/mL PAA10-CS concentration, 20 mg/mL PAA10-CS concentration, 0.1×PBS solvent, 10×PBS solvent, and a control group (no fluorescence). The samples were then cryosectioned and observed under a confocal microscope through a DAPI filter. The fluorescence was quantified from the images (area percent, MATLAB®) and significance (α=0.05) was determined using a one-way ANOVA with a Tukey post hoc test.

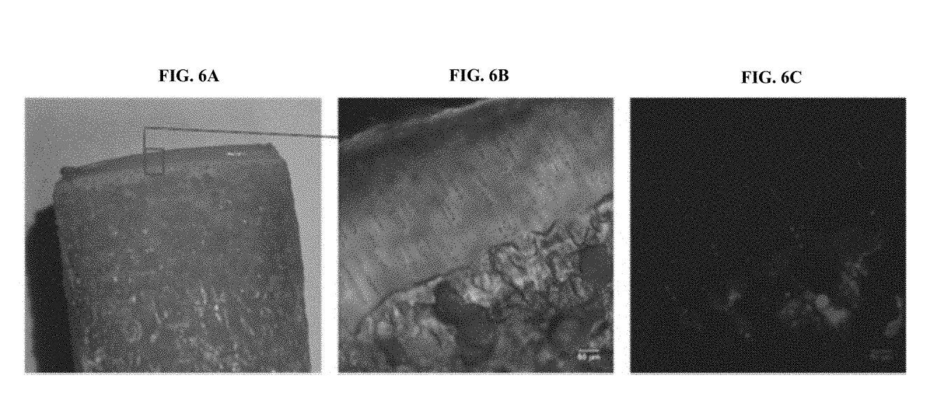

The results showed that, grossly, the fluorescently labelled PAA10-CS penetrated the cartilage surface and into the bony region of the plug (FIGS. 6A-6C). Confocal microscopy images of cryosections show normal cartilage morphology (contrast image) and infiltration of the cartilage and subchondral bony region with PAA10-CS and PAA250-CS.

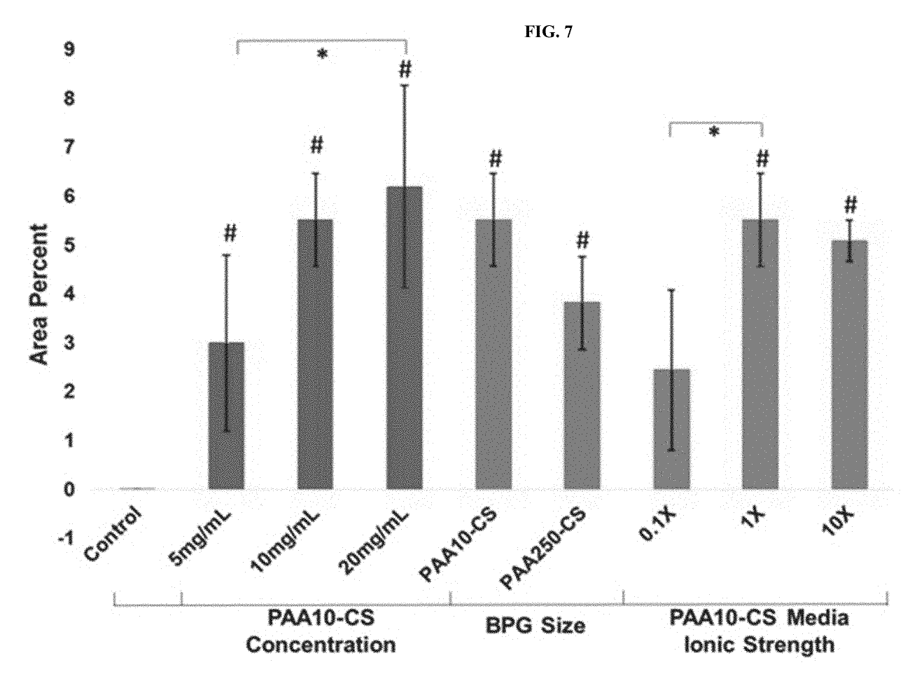

BPGs passively diffused into cartilage under all conditions, localizing around the chondrocytes after diffusing into the cartilage. Without wishing to be limited by any theory, this localization may be due to preferential binding to collagen VI, which has a higher concentration in the pericullar matrix surrounding the chondrocytes. The localization could also be due to the fact that the region has different concentrations of proteoglycans and glycosaminoglycans. Increasing the concentration of BPG solutions caused an increase in the amount of BPGs that diffused into the cartilage matrix (FIG. 7). Both sizes of molecules, PAA10-CS and PAA250-CS, were able to diffuse into the articular cartilage with the PAA250-CS having ˜30% less fluorescent area for the same time point.

The ion concentration was varied to determine whether the diffusion was affected by the charges present on the molecules and within the extracellular matrix. By increasing the ionic strength of the BPG solution, charge on the BPGs and in the matrix can be locally neutralized, allowing the molecules less repulsion when diffusion. This is consistent with the observation that the diffusion was somewhat hindered when 0.1×PBS BPG solutions were used.

Biomimetic proteoglycans (160-180 kDa and 1,600 kDa) were shown to diffuse through cartilage from surface exposure. The PAA250-CS is the largest known molecule shown to diffuse into cartilage, the former being a 500 kDa dextran. BPGs distributed throughout the depth of articular cartilage and localize around chondrocytes. Biomimetic proteoglycans can molecularly engineer normal cartilage and be used as a therapeutic treatment for degenerated cartilage.

### Example 4: Early Diffusion of Biomimetic Proteoglycans Results in Pericellular Augmentation Ex Vivo

### Methods

All BPG solutions were synthesized using previously described methods (Prudnikova K. et al. Biomacromolecules (2017)). Briefly, PAA10-CS comprises a 10 kDa PAA core with approximately 7-8 attached CS chains, giving the molecule a molecular weight of approximately 160-180 kDa, and PAA250-CS comprises a 250 kDa PAA core with ˜60 attached CS chains, giving the molecule a molecular weight of ˜1,600 kDa. BPGs and CS were fluorescently labeled with DCCH (7-Diethylaminocoumarin-3-Carboxylic acid, Hydrazide) (Stuart & Panitch, Biomacromolecules (2009)) and reconstituted in 1×PBS at 10 mg/mL. Osteochondral sections were cored from the femoral condyles of mature bovine knee joints from Animal Technologies, INC. The osteochondral sections were immersed in fluorescently labeled CS or BPG solutions such that the articular cartilage surface was submerged while limiting the amount of material which could diffuse through the cut lateral sides of the cartilage. The osteochondral sections were first equilibrated in 1×PBS then the articular surface was exposed to the PAA10-CS (10 kDa PAA with ˜7-8 attached CS, MW ˜160-180 kDa), PAA250-CS (250 kDa PAA with ˜60 attached CS, MW ˜1.6 MDa), and CS solutions for 5 min, 15 min, 30 min, and 1 hour (n=3). The osteochondral sections were then embedded in optimal cutting temperature compound (OCT) and cryosectioned. The sections were observed using an Olympus FLUOVIEW FV1000 confocal microscope through a DAPI filter to detect the DCCH fluorescence.

### Results

PAA10-CS, PAA250-CS, and CS molecules were able to passively diffuse through the articular surface into the extracellular matrix. FIG. 8 is a sequence of fluorescent images (top panels) with their counterpart contrast images (bottom panels) showing one PAA10-CS sample from each time point including control and 24 hour diffusion samples. The fluorescently labeled molecules are visible in the articular cartilage's superficial zone even with only 5 minutes of surface contact with the experimental solutions. At 15 and 30 min, the PAA10-CS molecules can be seen moving further into the middle zone of the articular cartilage. By 1 hour, PAA10-CS had diffused throughout the entire depth of the articular cartilage, similar to the 24 hour diffusion profile.

### Example 5: Biomimetic Proteoglycan Introduced into Ex Vivo Human Osteoarthritic Cartilage

Human osteochondral fragments were obtained from patients that underwent total knee arthroplasty (Drexel University IRB #1503003490). The osteochondral fragments from the tibial plateau were cut into 9×9 mm sections from both load bearing and non-load bearing regions to obtain samples with varying degrees of osteoarthritis. The Collins' method was utilized to grade the cartilage sections where grade 0 indicates non-osteoarthritic cartilage, grade 1 mild osteoarthritis, grades 2-3 intermediate osteoarthritis, and grade 4 severe osteoarthritis where the cartilage is essentially fully degraded. The human osteochondral sections were suspended in fluorescently labeled CS and biomimetic proteoglycan solutions such that the articular cartilage surface was submerged while limiting the amount of biomimetic proteoglycan solution that could enter through the cut lateral sides of the section. PAA10-CS molecules were synthesized as described in Example 1. The human osteochondral sections were equilibrated in 1×PBS and then immersed in fluorescently labeled 20 mg/mL CS and PAA10-CS solutions for 24 hours with care taken to expose only the articular surface to the solution. The osteochondral sections were then embedded in OCT and cryosectioned. The sections were observed using an Olympus FV1000 confocal microscope through a DAPI filter to detect the DCCH fluorescence.

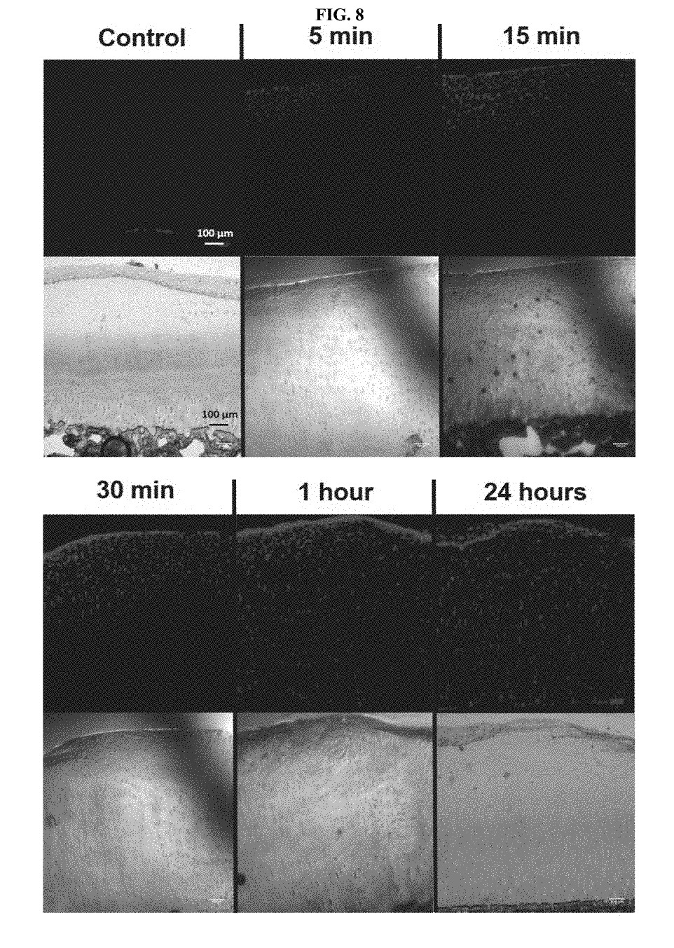

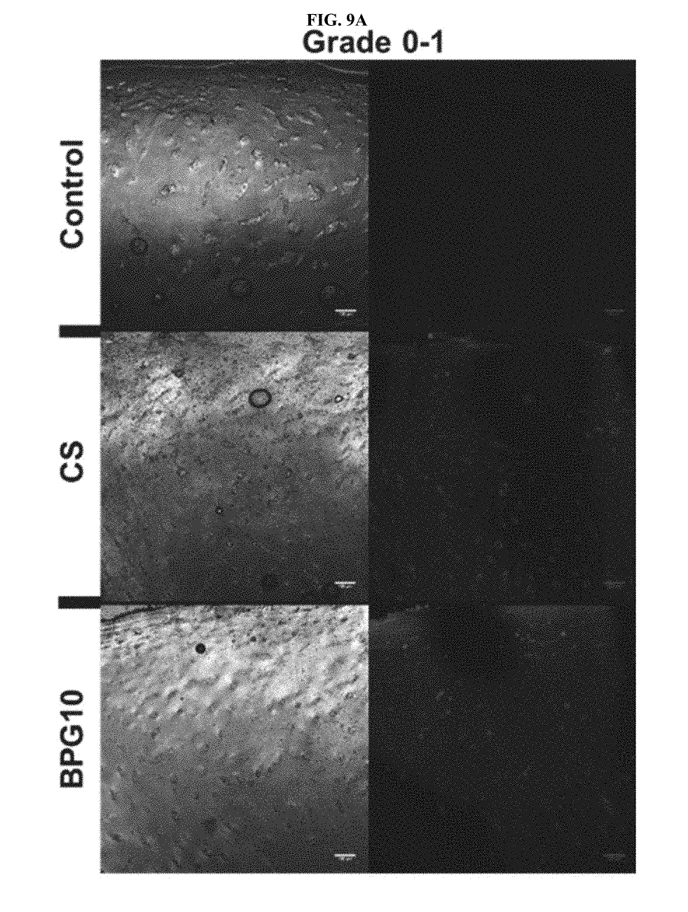

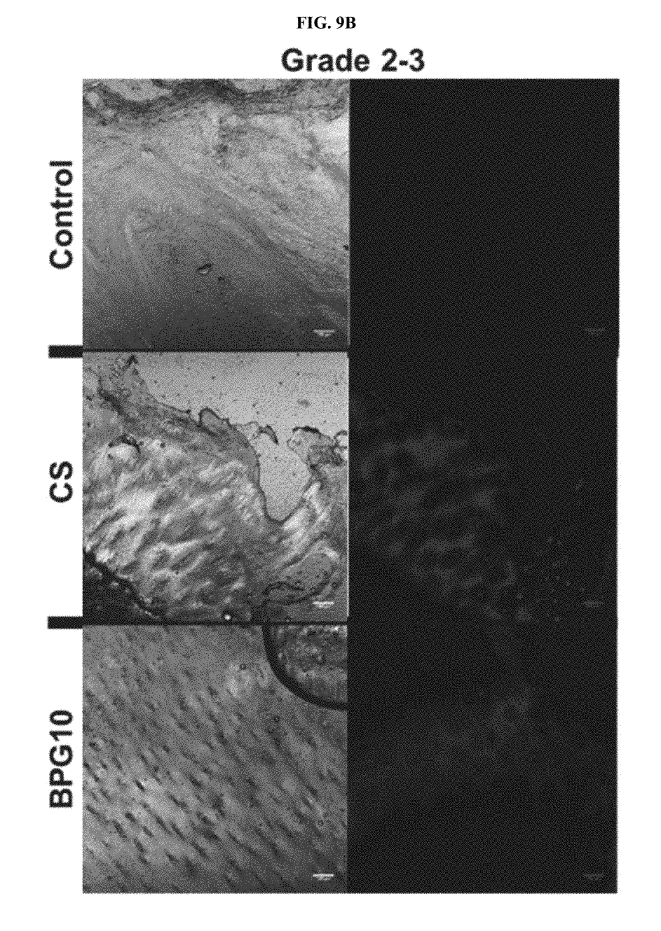

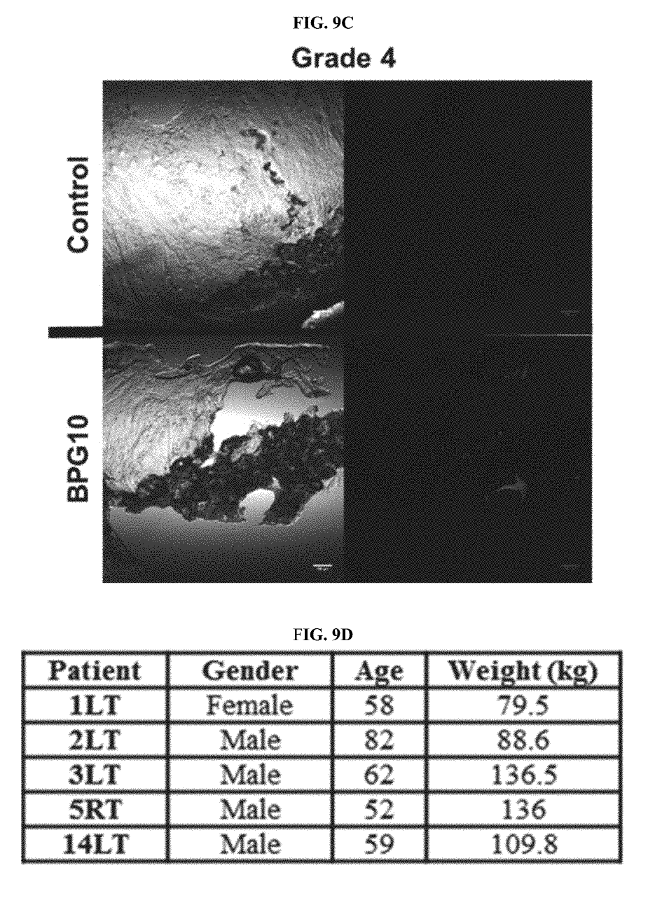

Both CS (22 kDa) and PAA10-CS (180 kDa) passively diffused into the human cartilage for grades 0-3 and dispersed throughout the cartilage extracellular matrix (FIGS. 9A-9C). The PAA10-CS and CS molecules tend to aggregate around the chondrocytes within the cartilage, as indicated by higher fluorescence intensity in the images. OA grades 0-1 showed more PAA10-CS and CS chondrocyte localization than grades 2-3. The localization of biomimetic proteoglycans and CS around chondrocytes and the overall diffusion into the cartilage extracellular matrix seems to decrease with more degenerative cartilage. Due to the insufficient cartilage in OA grade 4 samples, the diffusion experiments with these samples yielded more variable results. It was observed that biomimetic proteoglycans infiltrate into human articular cartilage with varying degrees of OA, distribute throughout the extracellular matrix, and localize around the chondrocytes in the pericellular region and inter-territorial zone.

### Example 6. Interaction of BPG with Collagen II

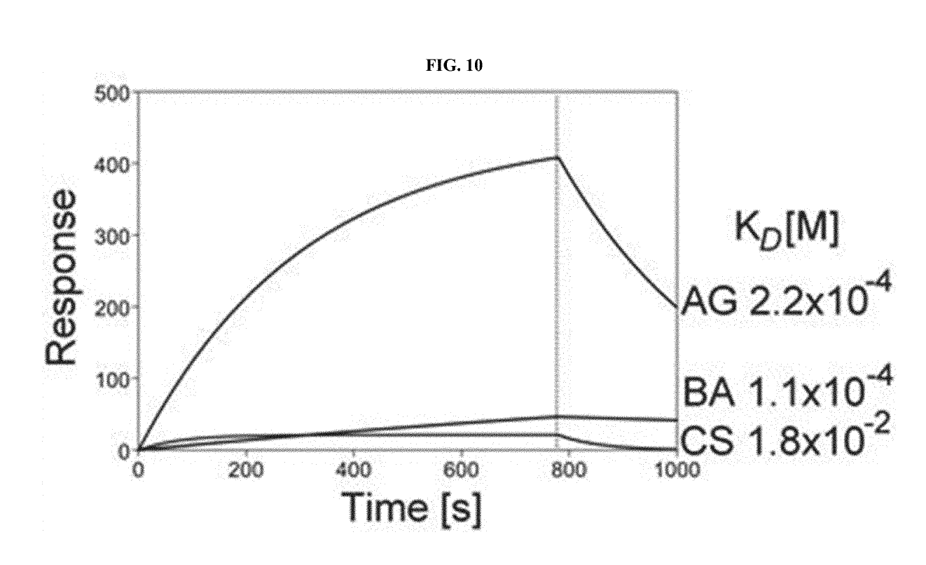

Intermolecular interactions drive the formation of complex architecture of the extracellular matrix of cartilage. An important element of this architecture is the network of collagen fibrils and aggrecans. To analyze the ability of PAA250-CS to bind collagen II, biosensor-based assays was employed. A biosensor measures molecular binding events in a real-time fashion. This instrument utilizes optical properties of intermolecular complexes to measure their accumulation on a sensor chip. In the assays, collagen II was covalently immobilized on a chip. Subsequently, PAA250-CS was added to a system in solution and then the “on” and the “off” rates were measured. Based on experiments done with various concentrations of PAA250-CS, the K_{D} value was calculated to characterize the collagen II-BPG binding interaction. Based on these measurements the binding affinity was calculated. The K_{D} of collagen II-aggrecan (2.2×10⁻⁴) was on the same order of magnitude as that of collagen II-PAA250-CS (1.1×10⁻⁴) (FIG. 10). In contrast, the K_{D} of collagen II-chondroitin sulfate interaction was two orders of magnitude lower (1.8×10⁻²). This indicates that there is a higher binding affinity (proportional to the inverse K_{D}) for collagen II for aggrecan and PAA250-CS as compared to chondroitin sulfate alone, likely due to the relatively high charge density associated with natural and biomimetic proteoglycans as compared to that of single chondroitin sulfate chains.

### Example 7: In Vivo Response to BPG Intra-Articular Injections Using an Arthritic Rabbit Model

The effect of BPG on the meniscus and knee joint articular cartilage is evaluated using an in vivo OA rabbit model. Fluorescently labelled BPG in a PBS carrier is injected into the knee joint (intra-articular) space, and its infiltration into cartilage is examined over time. Global joint pathology is evaluated using histology. Histology is compared OA-induced joints with no treatment and non-OA (normal) contralateral controls.

### Experimental Groups

Twenty-eight 20-24 week old, female New Zealand White Rabbit (Oryctolagus cuniculus) weighing approximately 3 kg each (Covance, Inc.) were randomly assigned to different experimental groups. The treatment of the rabbits was in accordance with approved IACUC protocol (#20349). Upon arrival all of the rabbits were given a week to acclimate. Six rabbits were used in a pilot with the right hind limb receiving surgery and the left serving as a normal control. Rabbits were then put into 4 groups: PAA10CS only (n=5), OA (n=5), OA+PAA10CS (n=5), OA+HA (EUFLEXXA®) (n=5) and the PAA10CS Fluorescent only (n=2). The left hind limb was used a contralateral normal control for all rabbits. Animal behavior was assessed throughout study by monitoring the animals' gait and eating habits.

### Anterior Cruciate Ligament Transection Surgery Procedure

Anterior cruciate ligament transection surgery (ACLT) (REFS) was used to promote the development of OA due to joint instability in the OA, OA+PAA10CS, and OA+HA (EUFLEXXA®) groups. Under sterile conditions, general anesthesia was administered along with an initial dose of 0.05 mg/kg buprenorphone subcutaneously to reduce the pain from the surgery for 6 hours. The rabbits' right knee was shaved, sterilized, and draped. A medial parapatellar incision was made, and the patella was dislocated allowing flexion on the hind limb. The ACL was transected and the joint space was irrigated with saline. Both the joint capsule and skin were sutured closed. Another dose of subcutaneous 0.05 mg/kg buprenorphone was given as needed for signs of pain or distress for up to 24 hours post-surgery. Both anterior-posterior and lateral x-rays were taken of the joint to ensure that no significant damage was done to the joint, although none was expected.

### Injection Procedure

Intra-articular injections were used to access the synovial space of the rabbits' knees. Rabbits in the PAA10CS only group received three injections of 50 mg/mL PAA10CS in 1×PBS spaced one week apart. Rabbits in the OA+PAA10CS and OA+HA groups received 3 injections starting 5 weeks post ACLT of 50 mg/mL PAA10CS in 1×PBS and HA (EUFLEXXA®), respectively, spaced one week apart. The Fluorescent group received only 1 injection of 50 mg/mL PAA10CS labeled with DCCH. All rabbits were euthanized 1 week following their last injection, 8 weeks following ACLT surgery for the OA group, and 5 days following fluorescently labeled PAA10CS injection for the Fluorescent group.

### Coefficient of Friction Testing

Post euthanasia, the hind limbs of all the rabbits were removed and all skin, muscle, and surrounding soft tissue was excised from each limb leaving the synovial capsule and ligaments intact. The femur was resected near the proximal end and the tibia was resected near the distal end. Both the femur and tibia were then embedded in a potting material and inserted into a custom built Stanton pendulum device (FIG. 11A) to measure the whole joints coefficient of friction. The neutral position of the joint was at a 135° angle, to simulate the typical sitting knee flexion angle for rabbits. The pendulum mass, including the suspended part of the frame, was 7 kg, designed to be approximately 2.5 times the rabbits body weight, a physiologically relevant load acting on the joint. The pendulum was allowed to oscillate at a frequency of ˜1 Hz to simulate a walking gait. The pendulum's oscillation was recorded and repeated 5-6 times for each hind limb. A custom MATLAB® code was used to analyze the pendulum motion and to obtain the peak angle decay (Δθ). A sample pendulum angle vs time plot from the code can be seen in FIG. 11B.

### Differential Equation Representing the Equation of Motion of the Pendulum

In determining the coefficient of friction of the joint, the equation of motion

$$I * \frac{{d}^{2} θ}{{dt}^{2}} + WL θ \pm {M}_{f} = 0$$

was used and air resistance was neglected. The moment due to air resistance is neglected (Mₐᵢᵣ). I is the moment of inertia of the pendulum about its axis. W is the weight of the pendulum. L is the distance from the pendulum center of gravity to the fulcrum. M_{f} is the moment due to friction about the pendulum axis of the joint serving as the fulcrum.

Imputing the peak angle decay (Δθ), the joint radius (r), and the distance between the fulcrum and center of gravity (L) into Stanton's equation

$$μ = \frac{L * Δθ}{4 r}$$

yields a joints coefficient of friction.

### Gross Pathology

Following the coefficient of friction testing, the rabbit joints were further dissected and photos were taken of the femoral condyles and tibial plateau. The gross pathology of the articular cartilage surfaces of both the tibia and femur were scored based on photographs by a blinded observer (XY). The cartilage samples were scored for cartilage integrity and osteophyte development based on a scale described by Cake, et al., Osteoarthritis Cartilage 2000, 8(6): 404-11. Cartilage integrity was scored 0-4: 0=normal, 1=roughened, 2=fibrillated, 3=erosions <5 mm, and 4=erosions >5 mm. Osteophyte development is scored 0-3: 0=none, 1=mild, 2=moderate, 3=marker osteophyte formation.

### Histopathology

The femoral condyles, tibial plateau, and menisci were fixed in 10% neutral buffered formalin for 72 hrs. Following fixation, the femoral condyles and the tibial plateau were decalcified in CAL-EX™, a chelating decalcifier in dilute hydrochloric acid, for 3 days. The samples were embedded in paraffin, sectioned and stained with Safranin-O, a stain for glycosaminoglycans (GAGs). The Mankin score was used to grade the articular cartilage samples for progression of osteoarthritis (Mankin, et al., J Bone Joint Surg Am, 1971, 53(3):523-37.). The score system used gave each rabbit limb a total score of 0-14 with 0 representing normal cartilage and is broken into the following categories: Cartilage Structure 0-6, Cellularity 0-3, Safranin-O Staining 0-4, and Tidemark Integrity 0-1. Synovial membrane attached to sides of the menisci was analyzed for markers of inflammation based the synovitis-score system developed by Krenn, et al., Pathol Res Pract. 2002, 198(5):317-25.

The synovitis scoring system gives each rabbit limb a score from 0-9 with 0 representing no inflammation present and is broken into the following categories: Hyperplasia 0-3, Inflammatory Infiltration 0-3, and Activation of Synovial Stroma 0-3. All samples were evaluated by a blinded pathologist.

### Fluorescent Imaging

Unstained articular cartilage sections from the Fluorescent group and normal controls were observed using a confocal microscope in order to detect any fluorescent BPGs present within the cartilage matrix.

### Statistical Analysis

The data presented here are presented as mean±standard deviation. All statistical analysis was performed using one-way analysis of variance followed by a Tukey post hoc test. P values less than 0.05 were considered significant for this study. All statistical analyses were perform using GRAPHPAD PRISM®.

### Perioperative Considerations and Behavior

No complications arose during any of the ACLT surgeries or intra-articular injections. Post-operative recover went smoothly with the animals returning to normal eating behavior within 48 hours and normal activity within one week on all hind limbs. The animals showed no adverse behavioral signs one week after the ACLT surgery and throughout the study duration. During joint dissection following friction testing, the joints were inspected to ensure transection of the anterior cruciate ligament and to ensure no other unintended damage had occurred. One rabbit from the HA (EUFLEXXA®) group was excluded due to its posterior cruciate ligament being damaged. X-ray images taken 5 weeks post ACLT surgery showed no detectable change in joint spacing when compared to x-rays taken before surgery.

### Gross Pathology

Gross pathological scoring of the cartilage by a blinded observer was largely variable within groups (Table 1). The gross scores ranged from 0.13 to 1.8 for the normal group and OA+PAA10-CS group, respectively. Given the scoring system described by Cake et al, all of the groups in this study fall within or below the mild OA range of the macroscopic OA scoring system with a scoring range from 0-7 with 0 representing normal articular cartilage and 7 representing complete cartilage erosion and large osteophyte presence (Cake, et al., Osteoarthritis Cartilage 2000, 8(6):404-11). The OA group (0.88±0.63) did not show significant gross changes when compared to the normal group (0.13±0.26). The groups where PAA10-CS, PAA10-CS only (1.1±1.34) and OA+PAA10-CS (1.8±1.04), was injected showed significant gross morphological changes when compared with the normal group. The OA+HA) (EUFLEXXA® (0.88±0.48) group had the same mean gross score as the OA group and was not significantly different than the normal group.

TABLE 1
Scores for Mankin (0-14), Synovitis (0-9), and Gross (0-7).

| | Category | | | | |
|---|---|---|---|---|---|
| | Normal | PAA10-CS only | OA | OA + PAA10-CS | OA + HA |
| Mankin Grading | n = 28 | n = 5 | n = 5 | n = 5 | n = 4 |
| Cartilage Structure | 2.32 ± 1.24 | 2 ± 1.41 | 3 ± 0 | 3 ± 0 | 3 ± 0 |
| Chondrocytes | 0.11 ± 0.31 | 0 ± 0 | 0 ± 0 | 0 ± 0 | 0 ± 0 |
| Safanin-O Staining | 0.96 ± 0.19 | 0.8 ± 0.45 | 1 ± 0 | 1 ± 0 | 1 ± 0 |
| Tidemark Integrity | 0 ± 0 | 0 ± 0 | 0 ± 0 | 0 ± 0 | 0.25 ± 0.5 |
| Mankin Score | 3.39 ± 1.4 | 2.8 ± 1.79 | 4 ± 0 | 4 ± 0 | 4.25 ± 0.5 |
| Synovitis Grading | n = 28 | n = 5 | n = 5 | n = 5 | n = 4 |
| Hyperplasia | 0.39 ± 0.49 | 1.6 ± 0.55 | 0.8 ± 0.45 | 1.2 ± 0.45 | 0.75 ± 0.5 |
| Inflammatory | 0.21 ± 0.5 | 2 ± 1.41 | 0.2 ± 0.45 | 1.8 ± 1.3 | 1 ± 0.82 |
| Infiltration | | | | | |
| Activation of | 0 ± 0 | 0.2 ± 0.45 | 0 ± 0 | 0.2 ± 0.45 | 0.25 ± 0.5 |
| Synovial Stroma | | | | | |
| Synovitis Score | 0.61 ± 0.79 | 3.8 ± 2.17*^{,a} | 1 ± 0.71 | 3.2 ± 1.92*^{,b} | 2 ± 1.63 |
| Gross Grading | n = 27 | n = 5 | n = 4 | n = 5 | n = 4 |
| Cartilage integrity | 0.09 ± 0.2 | 0.5 ± 0.5 | 0.88 ± 0.63 | 1.4 ± 0.65 | 0.88 ± 0.48 |
| Osteophyte | 0.04 ± 0.19 | 0.6 ± 0.89 | 0 ± 0 | 0.4 ± 0.42 | 0 ± 0 |
| Development | | | | | |
| Gross Score | 0.13 ± 0.26 | 1.1 ± 1.34^{c} | 0.88 ± 0.63 | 1.8 ± 1.04^{d} | 0.88 ± 0.48 |

All data is mean ± standard deviation.
*P < 0.001 vs Normal.
^{a}P < 0.01 vs OA.
^{b}P < 0.05 vs OA.
^{c}P < 0.05 vs Normal.
^{d}P < 0.001 vs Normal.

### Histopathology

Histological staining with safranin-O indicated a mildly arthritic or early degenerative state of the articular cartilage for the rabbit ACLT model used in this study. Representative images of the articular cartilage stained with safranin-O can be seen in FIGS. 12A-12E. The degenerative state of the model was too mild to show any statistical differences between groups when using the Mankin grading scale (0-14). Mankin score values can be seen in Table 1. The OA, OA+PAA10-CS, and the OA+HA group showed slightly more cartilage defects and lower GAG staining when compared to the unaltered joints of the normal group. The PAA10-CS only group (2.8±1.79), however, showed a lesser amount of cartilage degeneration and higher GAG staining than the normal group (3.39±1.4) resulting in the PAA10CS only group having the lowest Mankin score.

Examination of the synovial membrane tissue for inflammation markers showed mild to moderate inflammatory responses. The synovitis scores for all groups can be seen in Table 1. The OA (1±0.71) and OA+HA (2±1.63) group showed some mild inflammation, but were not statically higher than the normal group (0.61±0.79). The PAA10-CS only (3.8±2.17) and OA+PAA10-CS (3.2±1.92) groups both had more inflammation present when compared to the normal group or the OA group. The three groups that received injections of either PAA10-CS or HA (EUFLEXXA®) one week before euthanasia showed the most inflammation, although still in the mild to moderate stage. The pathologist who scored these sections also looked at the articular cartilage sections, and noted there were no dead or multinucleated giant cells within the articular cartilage, indicating no evidence of inflammation within the articular cartilage ECM.

### Fluorescent BPG Diffusion

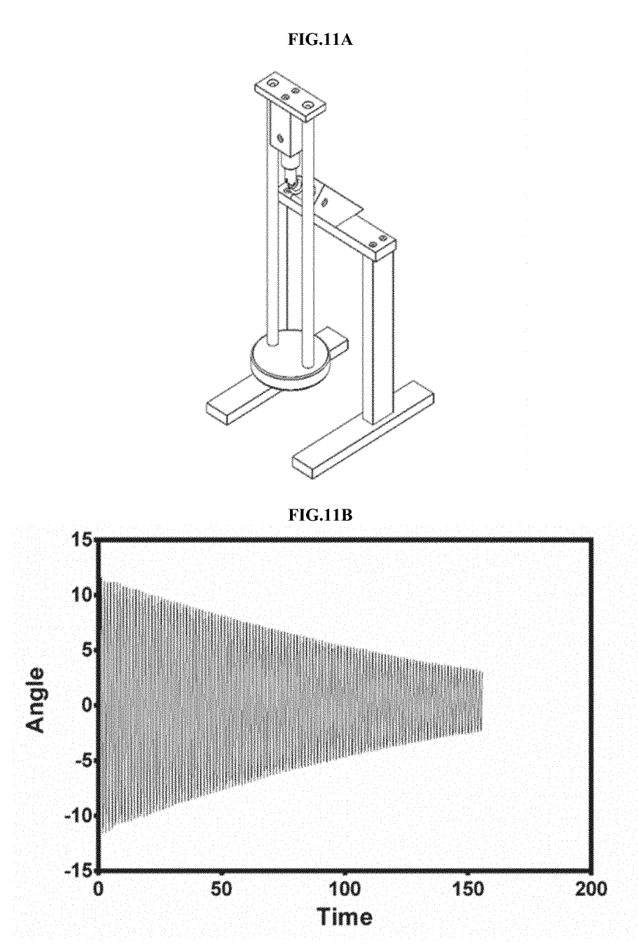

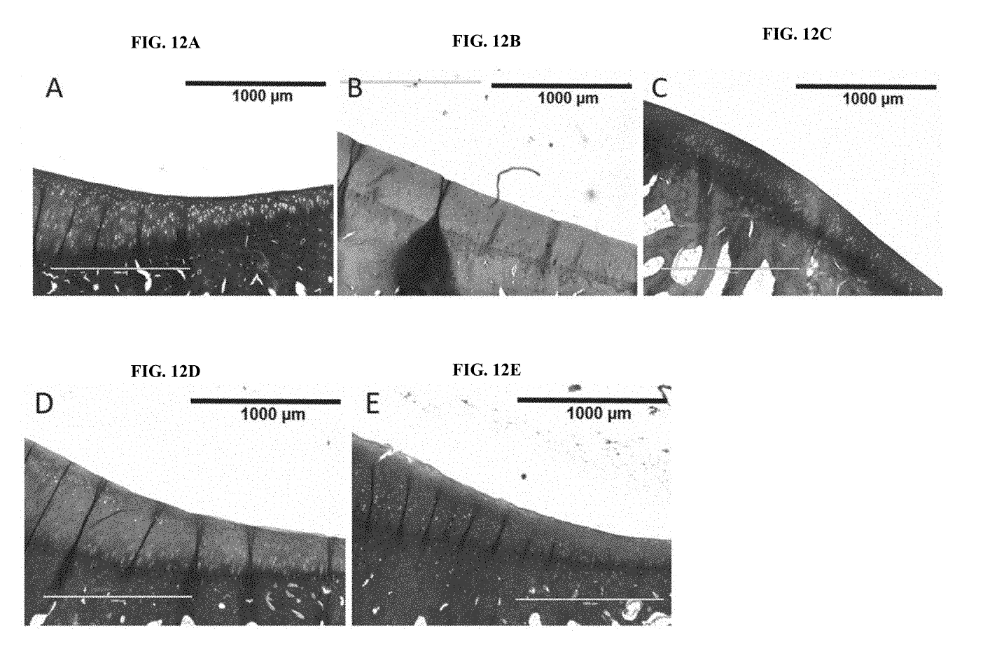

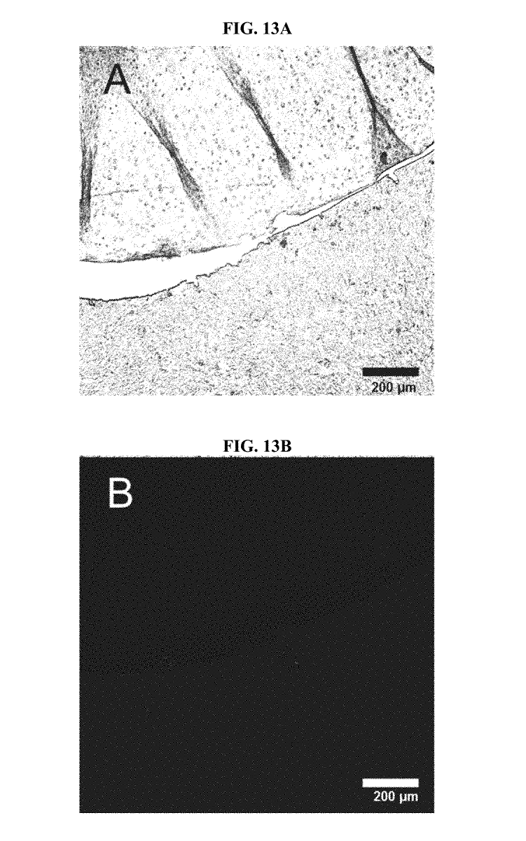

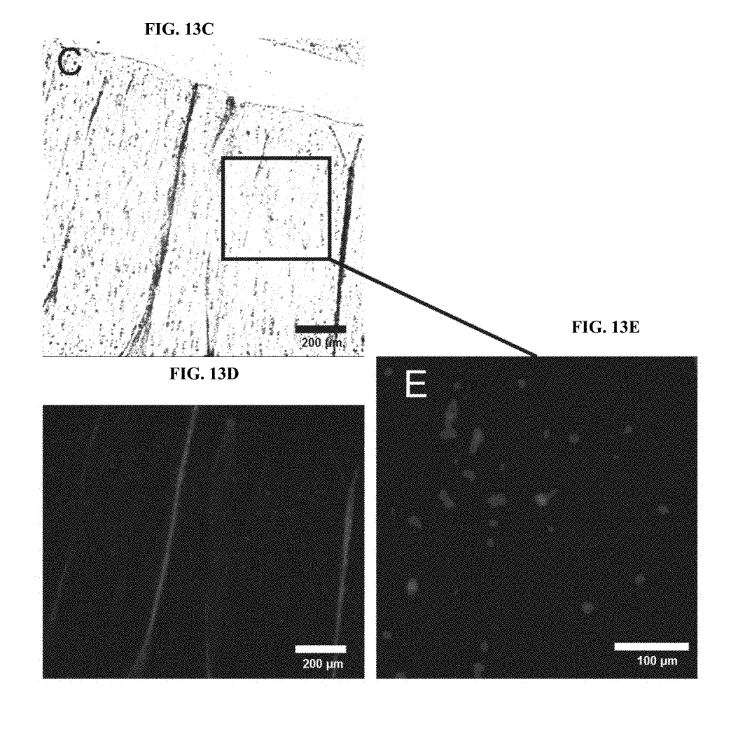

Confocal microscopy of unstained slides from the two rabbits that received one fluorescently labeled injection of PAA10-CS showed the BPG molecules diffused into the articular cartilage and were present five days after injection (the longest timepoint examined for this group). FIGS. 13A-13E show images of a non-injected control (FIGS. 13A-13B) and a cartilage that was injected with fluorescently labeled BPGs (FIGS. 13C-13E). The highlighted segments represent the fluorescence from the DCCH label on the BPG molecules and can be seen throughout the depth of the articular cartilage matrix. By zooming in on a particular region of the fluorescent image (FIG. 13E), the BPGs are observed especially localized near the chondrocytes within the articular cartilage.

### Joint Friction

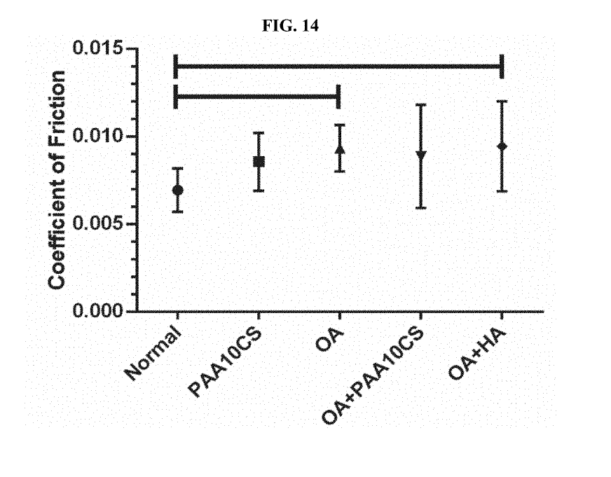

Coefficient of friction was determined for each joint tested in the custom-built pendulum. (FIG. 14). The OA group (0.0093±0.0013) showed a significantly higher coefficient of friction compared to normal control knee joints (0.0069±0.0012) mechanical evidence of cartilage degeneration. The OA+HA (EUFLEXXA®)(0.0094±0.0026) group also showed a significant increase in friction over the normal group. The PAA10-CS only (0.0085±0.0016) group and the OA+PAA10-CS (0.0089±0.0029) group did not have a significantly different friction coefficient from the normal or OA groups. The lack of significance between the PAA10-CS groups and the OA and normal groups is likely caused by the mild arthritis caused in the study not being severe enough.

The disclosures of each and every patent, patent application, and publication cited herein are hereby incorporated herein by reference in their entirety.

While the invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed:

1. A method of treating or preventing osteoarthritis (OA) in a mammal in need thereof, the method comprising contacting an articulating joint of the mammal affected by OA with a therapeutically effective amount of a biomimetic proteoglycan.

2. The method of claim 1, wherein the biomimetic proteoglycan is soluble in an aqueous solution.

3. The method of claim 1, wherein the articulating joint is at least one selected from the group selected from a hand joint, a wrist joint, a shoulder joint, an ankle joint, a knee joint, a hip joint, and a spine joint.

4. The method of claim 1, wherein the biomimetic proteoglycan comprises a core structure and at least one glycosaminoglycan (GAG), wherein the core structure and the GAG are covalently linked, and wherein the GAG is at least one selected from the group consisting of hyaluronic acid, chondroitin, chondroitin sulfate, heparin, heparin sulfate, dermatan sulfate, laminin, keratin sulfate, chitin, chitosan, acetyl-glucosamine, and oligosaccharides.

5. The method of claim 4, where the core structure is at least one selected from the group consisting of a synthetic polymer, a protein, a peptide, a nucleic acid, and a carbohydrate.

6. The method of claim 5, wherein the synthetic polymer is at least one selected from the group consisting poly(4-vinylphenyl boronic acid), poly(3,3′-diethoxypropyl methacylate), polyacrolein, poly(N-isopropyl acrylamide-co-glycidyl methacrylate), poly(allyl glycidyl ether), poly(ethylene glycol), poly(acrylic acid), poly(acryloyl chloride) and epoxides.

7. The method of claim 4, wherein the GAG is attached to the core structure through a linkage selected from the group consisting of a boronic acid-diol linkage, epoxide-amine linkage, aldehyde-amine linkage, carboxylic acid-amine linkage, sulfhydryl-maleimide linkage, acyl chloride-amine, and any combinations thereof.

8. The method of claim 7, wherein the GAG comprises at least one terminal handle selected from the group consisting of a primary amine, diol, and aldehyde.

9. The method of claim 1, wherein the biomimetic proteoglycan is resistant to the breakdown of an endogenous enzyme.

10. The method of claim 9, wherein the endogenous enzyme is at least one selected from the group consisting of hyaluronidases, aggrecanases and matrix metalloproteinases (MMPs).

11. The method of claim 1, wherein the biomimetic proteoglycan has at least one shape selected from the group consisting of cyclic, linear, branched, star-shaped, comb, graft, bottlebrush, dendritic, and mushroom.

12. The method of claim 1, wherein the biomimetic proteoglycan has at least one property that mimics a natural sulfated proteoglycan, wherein the at least one property is selected from the group consisting of water uptake and charge density.

13. The method of claim 1, wherein at least a fraction of the biomimetic proteoglycan migrates into the cartilage that is in contact with the joint.

14. The method of claim 13, wherein at least a fraction of the biomimetic proteoglycan migrates to the pericellular region or the inter-territorial zone of the cartilage.

15. The method of claim 13, wherein at least a fraction of the biomimetic proteoglycan remains intact within the cartilage for a minimum of five days.

16. The method of claim 1, wherein the biomimetic proteoglycan is crosslinked to itself and/or an additional molecule.

17. The method of claim 16, wherein the additional molecule is at least one selected from the group consisting of collagen, pectin, carrageenan, poly(L-lysine), gelatin, agarose, dextran sulfate, heparin, polygalacturonic acid, mucin, chondroitin sulfate, hyaluronic acid (HA), chitosan, alginate, alginate sulfate, poly(acrylic acid), poly(methyl methacrylate) (PMMA), poly(vinylsulfonic acid) (PVSA), poly(L-aspartic acid), poly(L-glutamic acid), poly(L-aspartic acid)-grafted-poly(ethylene glycol) (PAA-g-PEG), poly(L-glutamic acid)-grafted-poly(ethylene glycol) (PGA-g-PEG), poly(sodium 4-styrenesulfonate) (PSS), dermatan sulfate, and carboxymethyl cellulose (CMC).

18. The method of claim 1, wherein the articulating joint is further contacted with a therapeutic agent that treats OA.

19. The method of claim 18, wherein the therapeutic agent is at least one selected from the group consisting of acetaminophen, hyaluronic acid (HA), an anti-Fas antibody, a non-steroidal anti-inflammatory drug, a COX-2 inhibitor, a p21-activated kinase inhibitor, and an agent targeting promoting factors of cartilage reproduction.

20. The method of claim 1, wherein the mammal is human.
